(19) United States
(12) Patent Application Publication
(10) Pub. No.: US 2017/0231911 A1
(43) Pub. Date: Aug. 17, 2017

(54) METHODS AND COMPOSITIONS FOR IMPROVING OUTCOMES OF LIPOSOMAL CHEMOTHERAPY

(71) Applicant: Merrimack Pharmaceuticals, Inc., Cambridge, MA (US)

(72) Inventors: Elena GERETTI, Cambridge, MA (US); Bart S. HENDRIKS, Belmont, MA (US); Victor MOYO, Ringoes, NJ (US); Thomas WICKHAM, Groton, MA (US); Daryl C. DRUMMOND, Lincoln, MA (US); Dmitri KIRPOTIN, Revere, MA (US); Walid KAMOUN, Arlington, MA (US)

(21) Appl. No.: 15/357,462

(22) Filed: Nov. 21, 2016

Related U.S. Application Data
(63) Continuation of application No. 14/783,619, filed on Oct. 9, 2015, now abandoned.
(60) Provisional application No. 61/810,254, filed on Apr. 9, 2013.

(51) Int. Cl. A61K 9/127; A61K 9/00; A61K 31/704; C07K 16/32; A61K 31/664; A61K 31/282; C07K 16/40

(52) U.S. Cl. CPC A61K 9/1271; A61K 31/282; A61K 9/0019; C07K 16/40; C07K 16/32; A61K 31/664; A61K 31/704; C07K 2317/622

(57) ABSTRACT

Materials and methods for treating cancer patients with immunoliposomal chemotherapeutic agents are disclosed. The methods comprise administering to a patient a therapeutically effective amount of an immunoliposome in combination with a chemotherapeutic agent comprising an alkylating agent or an organoplatinum agent. The materials are immunoliposomal chemotherapeutic agents and chemotherapeutic preparations comprising an alkylating agent or an organoplatinum agent, each for use in the disclosed methods.

### CROSS-REFERENCE TO RELATED APPLICATIONS

This application is a continuation application of U.S. application Ser. No. 14/783,619, filed Oct. 9, 2015, which is a 35 U.S.C. §371(c) United States national phase application of PCT/US2014/033548, filed Apr. 9, 2014, which claims the benefit of and priority to U.S. Provisional Patent Application No. 61/810,254, filed Apr. 9, 2013, the entire contents of all of the referenced disclosures are hereby incorporated by reference in their entireties.

### BACKGROUND

Liposomes have proved a valuable tool for delivering various pharmacologically active molecules, such as anti-neoplastic (chemotherapeutic) agents, to cells, organs, or tumors. Liposome delivery has been shown to improve the pharmacokinetic profile and widen the therapeutic index of various anticancer drugs. Improved efficacy is in part a result of passive targeting of liposomes to tumor sites based on the enhanced permeability and retention (EPR) effect, whereby liposomes preferentially escape from the bloodstream into the tumor interstitium via leaky tumor vasculature and then become trapped in the tumor. To fully exploit this process, drug carriers should be engineered to retain drug while circulating, thereby preventing premature drug release before accumulating in the tumor but still allowing for release of drug once in the vicinity of the tumor. Antibody-targeted nanoparticles, such as immunoliposomes, e.g., targeted to a cell surface receptor, represent another strategy for more efficient delivery of chemotherapeutic agents to tumor cells.

It has been found, however, that deposition of liposomal drugs (including immunoliposomal drugs) in tumors varies. Tumors with higher drug deposition will, in general, have improved clinical outcomes. The degree to which liposomal particles can deposit into tumors has been shown to be highly variable in both preclinical tumor models and in clinical studies in which liposomes have been used as imaging agents to quantify the level and variability of tumor deposition. Increasing the magnitude and uniformity of liposomal drug deposition in tumors during treatment promises to improve patient outcomes. Therefore, there is an as yet unmet need to discover agents that will increase the magnitude and uniformity of liposomal drug deposition and to develop methods of using such agents to improve the efficacy of chemotherapeutic liposomes when administered to cancer patients. The present invention addresses this need and provides additional benefits.

### SUMMARY

Disclosed herein are methods and compositions for treating a cancer in a human patient, the methods comprising administering to the patient a combination therapy comprising administration of a preparation of immunoliposomes and administration of an alkylating agent or an organoplatinum agent. The combination therapy is optionally administered (or the composition are for administration) according to a clinical dosage regimen disclosed herein.

In one aspect, herein provided is a method of treating a cancer in a human patient, the method comprising at least one treatment cycle, each cycle comprising administration of an alkylating agent or an organoplatinum agent to the patient followed by administration of a therapeutically effective amount of an immunoliposome comprising an encapsulated chemotherapeutic agent and a plurality of externally oriented antibody molecules, wherein the administration of the immunoliposome is parenteral and is initiated from two to ten days after initiation of the administration of the alkylating agent or the organoplatinum agent; optionally wherein the administration of the immunoliposome is initiated before the administration of the alkylating agent or the organoplatinum agent is completed, optionally wherein the at least one cycle is two cycles, three cycles, four cycles or five cycles; and optionally wherein the antibody binds immunospecifically to a cell surface receptor on a human cell; optionally the alkylating agent is mechlorethamine, uramustine, melphalan, chlorambucil, ifosfamide, bendamustine, carmustine, lomustine, streptozocin, or busulfan; optionally the alkylating agent is cyclophosphamide; optionally the organoplatinum agent is cisplatin, oxaliplatin, satraplatin, picoplatin, nedaplatin, or triplatin; optionally the organoplatinum agent is carboplatin.

In one embodiment, the administration of the cyclophosphamide provides a dose of 100 mg/m² to 650 mg/m², optionally a dose of 150 mg/m², 200 mg/m², 250 mg/m², 300 mg/m², 350 mg/m², 400 mg/m², 450 mg/m², 500 mg/m², 550 mg/m², or 600 mg/m². In another embodiment the administration of the immunoliposome is initiated from three to six days after the administration of the cyclophosphamide is initiated. In yet another embodiment, the administration of the immunoliposome is initiated from four to five days after the administration of the cyclophosphamide is initiated.

In another embodiment, the administration of the cyclophosphamide is parenteral administration, optionally wherein the parenteral administration is intravenous, subcutaneous, intrathecal, intravesicular, or intramuscular administration and the cyclophosphamide is in an injectable solution. In another embodiment, the parenteral administration is a single intravenous administration. In another embodiment, the cyclophosphamide is in an oral dosage form and the administration of the cyclophosphamide is oral administration and the oral dose is from 1-5 mg/kg daily for 3-10 days.

In another embodiment, the antibody molecules bind immunospecifically to a human cell surface receptor that is a receptor tyrosine kinase. In another embodiment, the receptor tyrosine kinase is HER2. In yet another embodiment, the antibody molecules bind immunospecifically to a human cell surface receptor that is an ephrin receptor. In one aspect the ephrin receptor is EphA1, EphB1, EphB2, EphA3, EphB3, EphA4, EphB4, EphA5, EphA6, EphB6, EphA7, EphA8, EphA10. In another aspect the ephrin receptor is EphA2.

In another embodiment, the plurality of externally oriented antibody molecules consists of 10-200, 20-100, 30-75 or 40-50 scFv molecules. In another embodiment, the antibody molecules bind immunospecifically to a particular species of cell surface receptor on a human cell, and optionally upon binding of one or more of the antibody molecules to one or more receptors of the particular species on the human cell, the immunoliposome is internalized by the cell, optionally wherein the binding to the one or more receptors on the human cell is in vitro binding and the human cell is a cultured human cell.

In another embodiment, the particular species is selected from EGFR, HER2, ErbB3, ErbB4, FGFR1, FGFR2, FGFR3, FGFR4, FGFR6, IGF-1R, IGF-2R, EphA1, EphB1, EphA2, EphB2, EphA3, EphB3, EphA4, EphB4, EphA5, EphA6, EphB6, EphA7, EphA8, EphA10, c-Met, VEGFR-1, VEGFR-2, DDR1, IR, PDGFR-αα, PDGFR-αβ, PDGFR-ββ, TrkA, TrkB, TrkC, UFO, LTK, ALK, Tie-1, Tie-2, FZD1, FZD2, FZD3, FZD4, FZD5, FZD6, FZD7, FZD8, FZD9, FZD10, SMO, TLR1, TLR2, TLR3, TLR4, TLR5, TLR6, TLR7, TLR8, TLR9, TLR10, TLR11, TLR12, TLR13, PTK7, Ryk, CD3, CD4, CD8, CD28, TCR, NMDAR, LNGFR and MuSK.

In another embodiment, the encapsulated chemotherapeutic agent is selected from 2-chloro adenosine, 5-azacytosine, 5-azacytosine-arabinoside, 5′-deoxyfluorouridine, 5-FU, 5-imidodaunomycin, 6-mercaptopurine, allopurinol, aminoglutethimide, aminopterin, anastrozole, azathioprine, bicalutamide, bleomycin, bryostatin, busulfan, capecitabine, carboplatin, carcinomycin, carmustine, chlorambucil, cisplatin, cladribine, cyclophosphamide, cytosine arabinoside, dacarbazine, dactinomycin, daunorubicin, daunoryline mitoxantrone, deoxycytidine, didanosine, diethylstilbestrol, docetaxel cabazitaxel, doxorubicin, droloxifene, edatrexate, epirubicin, estradiol, etoposide, finasteride, fludarabine, fluorodeoxyuridine, flutamide, ftorafur, gemcitabine, gemcitabine, hydroxyurea, idarubicin, ifosfamide, irinotecan, leuprolide, lomustine, lurtotecan, mechlorethamine, medroxyprogesterone acetate, megesterol acetate, melphalan, methotrexate, mitomycin, mitotane, N-acetyladriamycin, N-acetyldaunomycine, ormaplatin, oxaliplatin, paclitaxel, pegaspargase, pentostatin, pentostatin, perfosfamide, pirarubicin, platinum-DACH, plicamycin, pyrimethamine, pyritrexim, rubidazone, rubidomycin, silatecan, streptozocin, streptozocin, tamoxifen, teniposide, testolactone, tetraplatin, thioguanine, thiotepa, thymitaq, tolmudex, topotecan, toremefine, trimethoprim, trimetrexate, trioxifene, trophosphamide, vinblastine, vincristine, vindesine, vinflunine, vinorelbine, vinpocetine, and zalcitabine.

### BRIEF DESCRIPTION OF THE DRAWINGS

FIGS. 1A-1D shows quantification of MM-302 (squares) and free doxorubicin (“free dox,” triangles) in tumor (FIG. 1A), heart (FIG. 1B), skin (FIG. 1C) and the ratio or tumor/heart (FIG. 1D) in tissues from a BT-474-M3 breast cancer mouse xenograft model. Quantification was measured as the % injected dose per gram of tissue was measured (% i.d./g).

FIGS. 2A-2D shows quantification of doxorubicin in tumor (FIGS. 2A-2C) and heart (FIG. 2B-2D) after administration with MM-302 alone or MM-302 following pretreatment with cyclophosphamide (MM-302+C). In FIGS. 2A and 2B, quantification was measured as the % injected dose per gram of tissue was measured (% i.d./g). FIGS. 2C and 2D show the areas under the curve (AUCs) with propagated error of the data in (2A) and (2B).

FIGS. 3A-3F shows cell characteristics of samples from tumor-bearing mice via tissue section analysis after dosing with cyclophosphamide. Shown is the percentage of H2AX—positive cells (FIG. 3A), the percentage of cleaved caspase 3-positive cells (Cl. Caspase 3 POS Cells, FIG. 3B), the percentage of cleaved PARP-positive cells (FIG. 3C), tumor cell density per area of the tumor in mm² (FIG. 3D), and % interstitial space area (FIG. 3E). Also shown are tissue sections from control cells and cells 96-hours after cyclophosphamide treatment showing tumor cells (the medium staining, red), non-tumor cells (the lightest staining, blue), and interstitial space (black) (FIG. 3F), showing that the cyclophosphamide-treated cell sample has significantly more interstitial space than the control cell sample, but non-tumor cells were unaffected.

FIGS. 4A-4E shows tissue sections from BT474-M3 tumor-bearing mice that were untreated or dosed with cyclophosphamide (C) 96 hr before injection of MM-302. DiI5-labelled MM-302 (MM-302-DiI5), doxorubicin, FITC-lectin labeled blood vessels and nuclei were imaged on frozen sections. Representative tumors are shown (FIG. 4A) as original images (top panels) and post-classification (bottom panels). The bottom right panel (MM-302+C) has a higher percentage of doxorubicin positive (DOX POS, purple) nuclei than the bottom left panel (MM-302 alone), which has DOX POS nuclei around the periphery of the tumor section only, while the interior of the tumor section is largely doxorubicin negative (DOX NEG, blue). Blood vessels are shown in green. The quantification of the percentage of doxorubicin positive nuclei is shown FIG. 4B. The % of γ-H2AX and cleaved caspase 3 positive cells is shown in (FIG. 4C). The cell sections show that tumor sections from control (no treatment, top left panel, or cyclophosphamide alone, bottom left panel) mice have a much smaller number of brightly lit cells that stain for γ-H2AX and cleaved caspase 3, while the cell section from animals treated with cyclophosphamide and MM-302 shows a significantly greater number of cells that stain for γ-H2AX and cleaved caspase 3. The results of quantification are shown in FIG. 4D for γ-H2AX and FIG. 4E for cleaved caspase 3.

FIG. 5A shows tumor volume in BT474-M3 tumor-bearing mice. Mice were untreated (open circle) or treated with MM-302 (open square), cyclophosphamide (open diamond), or a combination of the two agents, co-injected (solid triangle), or with cyclophosphamide (C) given 96 hr prior to MM-302 (solid square). Measurements are given as the % change in tumor growth relative to the first day of treatment (day 15).

FIG. 5B shows the % change in tumor growth at 96 hours relative to the first day of treatment (day 15). Bliss Independence Analysis was done at day 26. [[TGI Fractional]]

FIG. 6 shows that cyclophosphamide enhances tumor deposition of MM-302/doxorubicin regardless of the route of administration. Mice received no predose of cyclophosphamide (C) (open squares), 40 mg/kg C given i.p. 4 days before MM-302 (open diamonds), 80 mg/kg C given 4 days before MM-302 (open triangles), 170 mg/kg C given i.p. 4 days before MM-302 (solid diamonds), 170 mg/kg C given i.v. 4 days before MM-302 (solid triangles), or 20 mg C given by oral gavage daily for eight days (solid circles), the final dose being given on the first day of administration of MM-302.

FIG. 7 shows that tumors from mice that were pretreated with cyclophosphamide followed by administration of MM-302 had a significantly higher percentage of the injected dose of doxorubicin in the tumor tissue than mice treated with MM-302 alone. Mice were treated with MM-302 alone (open squares), untargeted liposome alone (PLD, open triangles), MM-302+C pretreatment (solid squares), or PLD+C pretreatment (solid triangles). Mice were sacrificed at various time points and the % injected dose per gram of tissue was measured (% i.d./g).

FIGS. 8A-8D shows that pre-dosing of cyclophosphamide enhances liposome deposition. FIG. 8A shows the tumor deposition in patients who were treated with ⁶⁴Cu-MM-302 with no pretreatment and patients who were additionally pre-treated with cyclophosphamide. Shown are data from PET/CT scans from a total of 12 patients who either received (solid shapes) or did not receive (open shapes) cyclophosphamide pretreatment. Scans 1, 2, and 3 were taken on days 1, 2 and 3 after MM-302 treatment, respectively. Tumor deposition was measured as the % injected dose per kilogram (% i.d./kg). FIG. 8B shows that the median tumor deposition on Days 2 and 3 in patients treated with cyclophosphamide (closed squares) was higher than in patients who did not receive cyclophosphamide (open squares). The overall tumor deposition median for each scan day was used to establish a pseudo-threshold to identify tumors (lesions) with low (<median) and high (≧median)⁶⁴Cu-MM-302 deposition (FIG. 8C). FIG. 8D shows the blood pharmacokinetics of patients with and without cyclophosphamide pretreatment, demonstrating that the increase in tumor deposition of the pretreated patients is not due to a difference in drug exposure between sets of patients.

FIGS. 9A-9B shows early assessment of response as measured by both change in tumor size (FIG. 9A) and progression free survival (FIG. 9B). Patients either received MM-302+ trastuzumab (“H”)—left side of panel, lighter bars) or pretreatment with cyclophosphamide (cyclo) followed by MM-302+ trastuzumab (right side of panel, darker bars).

FIGS. 10A-10C shows data indicating that pretreatment with carboplatin increases the deposition of targeted liposomes in tumors (FIG. 10A), but not in the liver (FIG. 10B) or spleen (FIG. 10C), in mouse xenograft models using a variety of cancer cell lines. Mice were pretreated with either carboplatin or saline (control) 96 hours prior to administration of a fluorescently-labeled unloaded (i.e., without encapsulate drug) EphA2 targeted immunoliposome. In the figures, for each cell line used in the xenograft study, the left hand bar of each pair of bars shows the mean fluorescence intensity of the saline-treated animals and the right hand bar of each pair shows the mean fluorescence intensity of the carboplatin-treated samples.

### DETAILED DESCRIPTION

Disclosed herein are combination therapies for use in treating a subject having a cancer, said therapies comprising treatment of the subject with a preparation of an immunoliposomal chemotherapeutic agent, and a sufficient amount of cyclophosphamide to increase the level of tumor deposition of the immunoliposomes.

As used herein, the term “about,” when modifying a numerical value, can be understood as within 10%, 9%, 8%, 7%, 6%, 5%, 4%, 3%, 2%, 1%, 0.5%, 0.1%, 0.05%, or 0.01% of the stated value.

The term “antibody” includes proteins with immunospecific binding characteristics comprising at least one immunoglobulin-derived antigen binding site (e.g., VH/VL region or Fv). For example, the antibody may be a human antibody, a humanized antibody, a bispecific antibody, or a chimeric antibody. The antibody may also be a Fab, Fab′2, scFv (single-chain variable fragment), SMIP, Affibody®, or a single domain antibody.

By “anthracycline” is meant a class of drugs derived from Streptomyces peucetius var. caesius that are used in cancer chemotherapy. Exemplary anthracyclines include, but are not limited to, daunorubicin, doxorubicin, epirubicin, idarubicin, mitoxantrone, and valrubicin.

By “compound” is meant any small molecule chemical compound, antibody, nucleic acid molecule, or polypeptide, or fragments thereof.

The term “doxorubicin” refers to the drug with the chemical name (8S,10S)-10-(4-amino-5 hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione. It is marketed under the trade names Adriamycin PFS®, Adriamycin RDF®, or Rubex®. Doxorubicin is an anthracycline antibiotic, closely related to the natural product daunomycin, and like all anthracyclines, it works by intercalating DNA. Doxorubicin is supplied in the hydrochloride form as a sterile red-orange lyophilized powder containing lactose and as a sterile parenteral, isotonic solution with sodium chloride and is also supplied as a sterile red-orange aqueous solution containing sodium chloride 0.9%. Doxorubicin is for IV use only. Doxorubicin has the following structural formula:

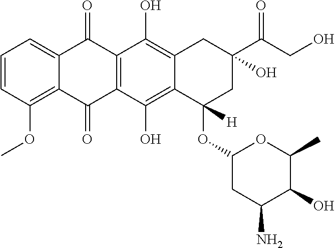

By “cyclophosphamide” is meant a synthetic antineoplastic drug with the chemical name 2-[bis(2-chloroethyl)amino]tetrahydro-2H-1,3,2-oxazaphosphorine 2-oxide monohydrate. Cyclophosphamide is marketed under the trade name CYTOXAN.

By “carboplatin” is meant cis-Diammine(1,1-cyclobutanedicarboxylato)platinum(II), which is marketed under the trade name PARAPLATIN. Like other organoplatinum antineoplastic agents, carboplatin interacts with DNA and interferes with DNA repair.

By “MM-302” is meant a unilamellar lipid bilayer vesicle of approximately 75-110 nm in diameter that encapsulates an interior aqueous space which contains doxorubicin in a gelated or precipitated state. The lipid membrane is composed of phosphatidylcholine, cholesterol, and a polyethyleneglycol-derivatized phosphatidylethanolamine in the amount of approximately one PEG molecule for 200 phospholipid molecules, of which approximately one PEG chain for each 1780 phospholipid molecules bears at its end an F5 single-chain Fv antibody fragment that binds to HER2. MM-302 is described (together with methods of making and using MM-302) in, e.g., PCT Patent Publication No. WO 2012/078695.

The term “therapeutically effective amount” refers to an amount of an agent that provides the desired biological, therapeutic, and/or prophylactic result. A therapeutically effective amount may be administered in one or more administrations. A therapeutically effective amount of a drug or composition is one that will: (i) reduce the number of cancer cells; (ii) reduce tumor size; (iii) inhibit, retard, slow to some extent, and/or stop cancer cell infiltration into peripheral organs; (iv) inhibit (i.e., slow to some extent and may stop) tumor metastasis; (v) inhibit tumor growth; (vi) prevent or delay occurrence and/or recurrence of tumor; and/or (vii) relieve to some extent one or more of the symptoms associated with the cancer.

In one embodiment, the compositions and methods disclosed herein are effective for treating patients with histologically or cytologically confirmed advanced cancer that is positive for HER2 (HER2⁺). HER2⁺ cancers are those in which the tumor cells overexpress HER2. A tumor that overexpresses HER2 is one that is identified as being HER2 “3+” or HER2 “2+” by immunohistochemistry (e.g., by HercepTest®), or gene-amplified positive by fluorescence in situ hybridization (FISH+). In some embodiments, a tumor may be HER2⁺ as determined by immunohistochemistry but negative for HER2 as determined by FISH. Chromogenic in situ hybridization (CISH) may also be used if FISH results are unavailable. Patients can be tested or selected for one or more of the above described clinical attributes prior to, during or after treatment.

As used herein, “cancer” refers to a condition characterized by abnormal, unregulated, malignant cell growth. In some embodiments, the cancer is a solid tumor e.g., a melanoma, a cholangiocarcinoma, clear cell sarcoma, or an esophageal, head and neck, endometrial, prostate, breast, ovarian, gastric, gastro-esophageal junction (GEJ), colon, colorectal, lung, bladder, pancreatic, salivary gland, liver, skin, brain or renal tumor. In other embodiments, the cancer is squamous cell cancer, small-cell lung cancer, non-small cell lung cancer, cervical cancer, or thyroid cancer. In certain aspects the solid tumor may be a HER2+ tumor.

### MM-302 Liposomes

“MM-302” refers to a HER2-targeted immunoliposome comprising the anthracycline chemotherapeutic agent doxorubicin. Immunoliposomes are antibody (typically antibody fragment) targeted liposomes that provide advantages over non-immunoliposomal preparations because they are selectively internalized by cells bearing cell surface antigens targeted by the antibody. Such antibodies and immunoliposomes are described, for example, in the following US patents and patent applications: U.S. Pat. Nos. 7,871,620, 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof”); U.S. Pat. No. 7,022,336 (“Methods for attaching protein to lipidic microparticles with high efficiency”); and U.S. Pat. Nos. 7,892,554 and 7,244,826 (“Internalizing ErbB2 antibodies.”). Immunoliposomes targeting HER2 can be prepared in accordance with the foregoing patent disclosures. Such HER2 targeted immunoliposomes include MM-302, which comprises the F5 anti-HER2 antibody fragment and contains doxorubicin. MM-302 contains, on average, 40-50 (about 45) copies of mammalian-derived F5-scFv (anti-HER2) per liposome.

An MM-302 liposome is a unilamellar lipid bilayer vesicle of approximately 75-110 nm in diameter that encapsulates an aqueous space that contains doxorubicin. The lipid membrane is composed of phosphatidylcholine, cholesterol, and a polyethyleneglycol-derivatized phosphatidylethanolamine in the amount of approximately one PEG molecule for 200 phospholipid molecules, of which approximately one PEG chain for each 1780 phospholipid molecules bears at its end an F5 scFv antibody fragment that binds immunospecifically to HER2.

TABLE 1
MM-302 Monotherapy Dosing

| | Dose 1 | Dose 2 | Dose 3 | Dose 4 | Dose 5 | Dose 6 | Dose 7 | Dose 8 | Dose 9 |
|---|---|---|---|---|---|---|---|---|---|
| Every | 10 | 15 | | | | | | | |
| week | mg/m² | mg/m² | | | | | | | |
| Every two | 10 | 15 | 20 | 25 | | | | | |
| weeks | mg/m² | mg/m² | mg/m² | mg/m² | | | | | |
| Every three | | 15 | 20 | 25 | 30 | 35 | 40 | | |
| weeks | | mg/m² | mg/m² | mg/m² | mg/m² | mg/m² | mg/m² | | |
| Every four | | | 20 | 25 | 30 | 35 | 40 | 45 | 50 |
| weeks | | | mg/m² | mg/m² | mg/m² | mg/m² | mg/m² | mg/m² | mg/m² |
| Every five | | | | | 30 | 35 | 40 | 45 | 50 |
| weeks | | | | | mg/m² | mg/m² | mg/m² | mg/m² | mg/m² |

MM-302 is administered as a monotherapy in the doses set forth in Table 1, above. In Table 1, “mg/m²” indicates mg of doxorubicin (formulated as MM-302) per square meter of body surface area of the patient. For MM-302, the dosing regimens indicated with an * are preferred. Dosing regimens may vary in patients with solid tumors that are “early” (pre-metastatic, e.g., adjuvant breast cancer) as compared to “advanced” (metastatic tumors). Preferred tumors are those in which the tumor cells overexpress HER2. A tumor that overexpresses HER2 is one that is identified as being HER2³⁺ or HER2²⁺ by HercepTest™, or HER2 FISH+ by fluorescence in situ hybridization. Alternatively, a preferred tumor that overexpresses HER2 is one that expresses an average of 200,000 or more receptors per cell, as quantified by the methods described in the Examples.

### Dosage and Administration of MM-302

MM-302 may be administered by IV infusion over 60 minutes on the first day of each 1-, 2-, 3-, 4-, or 5-week cycle. The first cycle Day 1 is a fixed day. Subsequent doses may be administered on the first day of each cycle ±3 days. Prior to administration, the appropriate dose of MM-302 must be diluted in 5% Dextrose Injection, USP. Care should be taken not to use in-line filters or any bacteriostatic agents such as benzyl alcohol.

MM-302 may be administered at a dose that ranges from about 100 mg/m² to about 1 mg/m². In other embodiments, MM-302 may be administered at a dose that ranges from about 50 mg/m² to about 2 mg/m². In other embodiments, MM-302 may be administered at a dose that ranges from about 40 mg/m² to about 3.22 mg/m². In still other embodiments, MM-302 may be administered at a dose of 60 mg/m², 55 mg/m², 50 mg/m², 45 mg/m², 40 mg/m², 35 mg/m², 30 mg/m², 25 mg/m², 20 mg/m², 16 mg/m², 14 mg/m², 12 mg/m², 10 mg/m², 8 mg/m², 6 mg/m², 4 mg/m², and/or 3.2 mg/m². In another embodiment, MM-302 may be administered at a dose of 50 mg/m², 40 mg/m², 30 mg/m², 16 mg/m², or 8 mg/m².

Pretreatment with or concomitant use of anti-emetics may be considered according to institutional guidelines. The actual dose of MM-302 to be administered is determined by calculating the patient's body surface area at the beginning of each cycle. A ±5% variance in the calculated total dose can be permitted for ease of dose administration.

### Pharmaceutical Compositions

Pharmaceutical compositions of immunoliposomes suitable for administration to a patient are preferably in liquid form for intravenous administration.

In general, compositions provided herein typically comprise a pharmaceutically acceptable carrier. “Pharmaceutically acceptable” means a carrier that is approved by a government regulatory agency listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water or aqueous saline solutions and aqueous dextrose and glycerol solutions. Liquid compositions for parenteral administration can be formulated for administration by injection or continuous infusion. Routes of administration by injection or infusion include intravenous, intraperitoneal, intramuscular, intrathecal and subcutaneous. In one embodiment, both MM-302 and an anti-HER2 antibody (e.g., trastuzumab) are administered intravenously (e.g., separately or together over the course of a predetermined period of time, e.g., one hour).

MM-302 for intravenous infusion (e.g., over the course of one hour) is supplied as a clear liquid solution in sterile, single-use vials containing 10.1 ml of MM-302 at a concentration of 25 mg/ml in 20 mM histidine, 150 mM sodium chloride, pH 6.5, which should be stored at 2-8° C.

### Combination Therapy

According to the techniques disclosed herein, an alkylating agent or an organoplatinum agent may be used as a tumor priming agent to be administered in combination with an immunoliposome, e.g., MM-302, in order to effect improvement in a cancer patient. When used in such combinations, the tumor priming agent increases levels of deposition of the immunoliposome in tumors. Surprisingly, as demonstrated in xenograft animal models, the increased levels of deposition are greater than those obtained in matched tumors with the same tumor priming agent and a matched liposome that differs from the immunoliposome in that it lacks antibody molecules.

As used herein, combined administration (co-administration) may include simultaneous administration of the compounds in the same or different dosage form, or separate administration of the compounds (e.g., sequential administration of cyclophosphamide and MM-302). For example, a tumor priming agent, e.g., cyclophosphamide or carboplatin, can be administered in combination with the immunoliposome, wherein both the tumor priming agent and immunoliposome are formulated for separate administration and are administered sequentially. As such, the tumor priming agent may be administered first, followed by administration of the liposomal anti-cancer agent. In one embodiment, a patient is pre-treated with a tumor priming agent that is cyclophosphamide prior to treatment with a liposomal anti-cancer agent.

In one embodiment, the tumor-priming agent, e.g., cyclophosphamide or carboplatin, is co-administered with the immunoliposome. In another embodiment, the tumor-priming agent is administered about one day, about two days, about three days, about four days, about five days, about six days, about seven days, about eight days, about nine days, or about ten days before the administration of the immunoliposome.

### Treatment Protocols

Suitable treatment protocols include, for example, those wherein (A) the immunoliposome (e.g., MM-302) may be administered to a patient (i.e., a human subject) once per every three weeks over a course of, e.g., fourteen three-week cycles (at a dose of 30-50 mg/m² per cycle) and (B) the tumor priming agent (e.g., an alkylating agent or organoplatinum agent such as cyclophosphamide or carboplatin) is administered to a patient once every three weeks over a course of at least the first four of fourteen three-week cycles, and the tumor priming agent is administered prior to the immunoliposome.

In another embodiment, the immunoliposome is administered once every three weeks or once every four weeks. The administration cycle may be repeated, as necessary.

In the preceding embodiments, the tumor priming agent may be administered one day, two days, three days, four days, five days, six days, or seven days prior to the administration of the immunoliposome. In one embodiment, the tumor priming agent is cyclophosphamide and is administered daily on each day between the first administration of the cyclophosphamide and the first administration of the immunoliposome.

### Kits and Unit Dosage Forms

Also provided are kits that include, in a container, a pharmaceutical composition containing an immunoliposome (e.g., MM-302), and a pharmaceutically-acceptable carrier, which composition is adapted for use in the preceding methods. The kits may optionally also include instructions, e.g., comprising administration schedules, to allow a practitioner (e.g., a physician, nurse, or patient) to administer the compositions contained therein to a patient having a cancer, either alone or in combination.

Optionally, the kits may include multiple packages of the single-dose pharmaceutical composition(s) containing an effective amount of the tumor priming agent (e.g., cyclophosphamide) and/or an effective amount of an immunoliposome (e.g., MM-302) for a single administration or a combination administration in accordance with the methods provided above. Optionally, instruments or devices necessary for administering the pharmaceutical composition(s) may be included in the kits. For instance, a kit may provide one or more pre-filled syringes containing an immunoliposome in an amount sufficient for administration in the above methods.

The following Examples are merely illustrative and should not be construed as limiting the scope of this disclosure in any way as many variations and equivalents will become apparent to those skilled in the art upon reading the present disclosure.

### Examples

Materials and Methods Used in these Examples:

Materials:

Cyclophosphamide monohydrate (cat #C0768), Doxorubicin hydrochloride (cat # D1515) and human insulin are from SIGMA-ALDRICH, Inc. (St. Louis, Mo.). FITC-conjugated lectin (lycopersicon esculentum (tomato) lectin, Cat # FL-1171) is purchased from Vector Laboratories, Inc. (Burlingame, Calif.). Acetic acid, methanol, and acetonitrile are from EMD Chemicals Inc. (Gibbstown, N.J.). Water and trifluoroacetic Acid (TFA) are from J. T. Baker (Phillipsburg, N.J.). HOECHST® 33342 trihydrochloride trihydrate, ProLong Gold®, and DiIC18(5)-DS (DiI5) are from Invitrogen (Carlsbad, Calif.). Cholesterol and 1,2-distearoyl-sn-glycero-3-phosphoethanolamine-N-[amino(polyethylene glycol)-2000] (ammonium salt) (PEG-DSPE) are from Avanti Polar Lipids Inc. Hydrogenated soy phosphatidylcholine (HSPC) is from Lipoid (Newark, N.J.). Fetal bovine serum (FBS) (cat#16140-071) is from Tissue Culture Biologicals. RPMI, MEM, Leibovitz's, DMEM and F12 media are from Gibco (Invitrogen). Trypsin-EDTA (0.25%, cat #25200-056), geneticin and penicillin G/streptomycin sulphate mixture are from GIBCO (Invitrogen). Estrogen pellets (0.74 mg; 60-day release) are from Innovative Research of America (cat # SE-121, Sarasota, Fla.). Hoechst® 33342 trihydrochloride trihydrate (cat #H1399), ProLong® Gold (cat #P36934), and DiIC18(5)-DS (DiI5) are from Invitrogen (Carlsbad, Calif.). Goat anti-mouse Alexa Fluor® 555 and goat anti-rabbit Alexa Fluor® 555 are from Molecular Probes (Eugene, Oreg.). Goat anti-mouse Alexa Fluor® 488, rabbit anti-human Cleaved Caspase 3 (cat #9661), rabbit anti-human Cleaved PARP, and SignalStain® Antibody diluent cat #8112) are from Cell Signaling Technology. Goat anti-hamster (Armenian) Alexa Fluor® 647 is from Jackson Immunoresearch. Armenian hamster anti-human CD31 and mouse anti-human phospho-Histone H2AX are from Millipore. Mouse anti-human cytokeratin (cat # M351501-2), rabbit anti-cow cytokeratin, mouse anti-human Ki67, EnVision+™ System-HRP labeled Polymer anti-rabbit (cat #K4003), and EnVision+ System-HRP labeled Polymer anti-mouse (cat #K4001) are from DAKO (Carpinteria, Calif.). Cyanine 5 Tyramide is from Perkin Elmer (cat # SAT705A, Boston, Mass.). Rabbit anti-human p27 KIP1 is from Abcam Inc. (Cambridge, Mass.). Rabbit anti-human HER2 is from Thermo Scientific (cat #RM-9103S 1).

Preparation of Immunoliposomes:

Liposomes are prepared and loaded with doxorubicin using an ammonium sulfate gradient as previously described (Kirpotin et. al., Cancer Res. 2006; 66:6732-40; Park et al., Clin Cancer Res. 2002; 8:1172-81). The lipid components are HSPC, cholesterol, and PEG-DSPE (3:2:0.3, mol:mol:mol). The anti-ErbB2 (F5)-PEG-DSPE conjugate is prepared and inserted into the liposome to form immunoliposomes as reported by Nellis et al., (Biotechnol Prog. 2005; 21:205-20; Biotechnol Prog. 2005; 21:221-32). The DiI-5-labelled liposomes, MM-302-DiI5 and PLD-DiI5, are prepared as above with the difference that the DiIC18(5)-DS (DiI5) dye is solubilized with the lipid components at a concentration of 0.3 mol % of total phospholipid. In all cases unloaded free doxorubicin is removed using a Sephadex® G-75 size exclusion column eluted with Hepes buffered saline (pH 6.5). F5-lipo-DiI5 is prepared in a similar fashion as above but without doxorubicin, and incorporating an aqueous solution of HEPES buffered saline (pH 6.5).

Cell Culture:

BT474-M3 cells are grown in RPMI medium containing 10% FBS and 1% penicillin G/streptomycin sulphate. MDA-MB-453 are grown in Leibovitz's medium complemented with 20% FBS and 1% penicillin G/streptomycin sulfate. MCF-7 HER2 cells are cultured in MEM supplemented with human insulin (10 μg/ml), geneticin (1 mg/ml), 10% FBS and 1% penicillin G/streptomycin sulfate. Calu-3 are cultured in DMEM media supplemented with 20% FBS and 1% penicillin G/streptomycin sulfate.

Animal Studies:

As used herein, “s.c.” is subcutaneous administration, “i.v.” is intravenous administration, and “i.p.” is intraperitoneal administration. 7-week-old female NCR/nu nude mice are purchased from Taconic (Hudson, N.Y.) and 7-week old nu/nu mice are purchased from Charles River Laboratories (Wilmington, Mass.). In accordance with the PHS Policy & the Guide for the Care and Use of Laboratory Animals, all resident colony animals received acceptable standards in their care, use and treatment. The care and treatment of experimental animals is in accordance with Institutional Animal Care and Use Committee (IACUC) guidelines.

Establishment of Xenografts and Dosing: To Establish Tumors:

NCR/nu mice are inoculated with 15×10⁶ BT474-M3 cells (into the mammary fat pad, in 50 μl media), 20×10⁶ MDA-MB-453 (s.c. into the right flank of the mouse in 100 μl of media) or 10×10⁶ MCF-7 HER2 cells (into the mammary fat pad, in 50 μl media). 7-week-old nu/nu mice are inoculated with 10×10⁶ Calu-3 cells (s.c. into the right flank of the mouse in 100 μl of media). When tumors reached an average volume of 200-300 mm³, mice are pre-dosed or not with cyclophosphamide (i.p. 170 mg/kg) at different time points as indicated. Mice are subsequently dosed with DiI5-labeled anthracycline-loaded anti-HER2 immunoliposome-targeted liposomal doxorubicin (3 mg/kg), DiI5-labelled liposomal doxorubicin PLD (3 mg/kg), or free doxorubicin all at (3 mg/kg). Following liposomes or free doxorubicin injection (6 hr to 168 hr, as indicated), and before sacrificing, mice received 200 μl of FITC-lectin (i.v.) to label the vasculature. The lectin is let circulate for 5 min before sacrificing the mice in a CO₂ chamber. Immediately after respiratory arrest, the heart of the mouse is exposed by incision of the thorax and 20 mL of PBS is flushed through the left ventricle to remove the liposome still in circulation.

Tissue Collection:

Tumor, heart and the dorsal skin are collected and frozen at −80° for further HPLC doxorubicin quantification. In addition, a portion of the tumors and hearts is collected for histology, frozen and formalin-fixed paraffin-embedded (FFPE). For frozen sections, tumors and hearts are frozen in OCT compound in liquid nitrogen and stored at −80° C. until processing (10 μm-thick tissue slices). For FFPE sections, tumors are fixed in neutral buffered formalin for 24 hr followed by fixation in 70% ethanol until processing (5 μm thickness).

Tumor Growth Inhibition:

after BT474-M3 tumor establishment (a mean volume of approximately 320 mm³), mice are randomized into the following treatment groups (n=10/group) comprising animals receiving: PBS (control), anthracycline-loaded anti-HER2 immunoliposome, PLD, free doxorubicin (all at 3 mg/kg, i.v., n=3 total doses, q7d, day 19, 26 and 33 post-inoculation), cyclophosphamide (170 mg/kg, i.p. q14d, n=2 total doses, day 15 and day 29). Three additional groups will receive a combination of cyclophosphamide (170 mg/kg, i.p.) dosed 96 hr before the first and the third dose of anthracycline-loaded anti-HER2 immunoliposome, PLD or free doxorubicin, all at 3 mg/kg i.v. An additional group receives a combination of cyclophosphamide (170 mg/kg, i.p.) simultaneously with the first and the third dose of HER2-targeted liposomal doxorubicin (3 mg/kg i.v.). Tumor growth is monitored by caliper measurement twice each week. Tumor volumes are calculated using the formula: width²×length×0.52. Mice are weighed twice each week to monitor weight loss.

Pharmacokinetic Study (PK):

after BT474-M3 tumor establishment (mean volume of 250 mm³), mice are randomized into seven treatment groups (n=5/group) that receive PBS (control), anthracycline-loaded anti-HER2 immunoliposome or PLD (both at 3 mg/kg, i.v.). Two additional groups receive a combination of cyclophosphamide (170 mg/kg, i.p.) dosed 96 hr before anthracycline-loaded anti-HER2 immunoliposome or PLD, respectively. Two additional groups receive a combination of cyclophosphamide (170 mg/kg, i.p.) dosed simultaneously with anthracycline-loaded anti-HER2 immunoliposome or PLD, respectively. Blood samples are collected at 5 min, 30 min, 2 hr, 6 hr, and 24 hr post liposome dose. Blood is spun down for 5 min at 5000× and plasma is stored at −80° C. until analysis by HPLC.

Quantification of Doxorubicin within Tissues by HPLC:

Tumors, hearts and dorsal skin are weighed and minced. 1 mL H2O is added and tissues are disaggregated using a TissueLyser® (Qiagen). 900 μl of 1% acetic acid in methanol is added to 100 μl of the homogenate, lysates are then vortexed for 10 sec and placed at −80° C. overnight. Samples are spun at RT for 10 min at 10,000 RPM. Supernatants and doxorubicin standards are analyzed by HPLC (Dionex) using a C18 reverse phase column (Synergi Polar-RP 80A 250×4.60 mm 4 μm column). Doxorubicin is eluted running a gradient from 30% acetonitrile; 70% 0.1% trifluoroacetic acid (TFA)/H2O to 55% acetonitrile; 45% 0.1% TFA/H₂O during a 7 min span at a flow rate of 1.0 ml/min. The doxorubicin peak is detected at 6.9 min using an in-line fluorescence detector excited at 485 nm, and emitting at 590 nm. The extraction efficiency of doxorubicin from tumor, heart and skin tissues is estimated using internal control tissues spiked with known amounts of doxorubicin, and sample readings are corrected to account for the extraction efficiency.

Histology:

The liposomes (DiI5-fluorescent labeled), doxorubicin and perfused vessels (FITC-lectin labelled) signals are imaged on unfixed 10-μm thick frozen tumor and heart tissue sections. Slides are air-dried and mounted with ProLong® Gold with Hoechst® stain to counterstain nuclei. Cleaved caspase 3, γ-H2AX, cleaved PARP and cytokeratin stainings are performed on FFPE-sections (5 μm thick). After deparaffinization and rehydration, heat-mediated antigen retrieval is performed on a in a pre-treatment module (Thermo Scientific, Waltham, Mass.) in citrate buffer (pH 6) for 25 min at 102° C. After antigen retrieval, slides are stained on a Lab Vision Autostainer® 360 (Thermo Scientific). Endogenous peroxidase activity is blocked with Peroxidazed® 1 (10 min at RT) followed by a washing step with TBST and a protein blocking step with Background Sniper (10 min at RT). Next, slides are incubated with the primary antibodies diluted in Da Vinci Green or Signal Stain® Antibody diluent for 1 hr at room temperature (RT). After washing, slides are incubated with secondary antibodies for 30 min at RT Antibodies used are goat anti-mouse Alexa Fluor® 488 and goat anti-rabbit Alexa Fluor® 647, diluted in Da Vinci Green, for the γ-H2AX and cleaved caspase 3 antibodies, respectively; and EnVision®+ System-HRP labeled Polymer anti-rabbit (for the signal amplification of the cleaved PARP signal) containing the secondary antibody for cytokeratin (goat anti-mouse Alexa Fluor® 555) for the cleaved PARP and cytokeratin double staining. After washing, for the signal amplification of cleaved PARP, samples are incubated with TSA™ Cyanine 5 Tyramide Reagent for 10 min at RT. Slides are washed and counterstained with Hoechst followed by a wash step and mounting with ProLong® Gold. Slides are imaged on an Aperio® FL slide scanner at 20× magnification.

Image Analysis:

Images are analyzed using rulesets written in Definiens® Developer XD 2 (Definiens, Munich, Germany). Images are analyzed as full tumor or heart tissues. The % of doxorubicin-positive nuclei is determined via analysis of frozen tumor and heart sections. Following an initial tissue from background separation, nuclei are segmented based on the Hoechst® staining signal and they are designated doxorubicin-positive or negative classes based on the intensity of the doxorubicin signal within the nucleus. The distribution of the liposomes relative to the vasculature is quantified as follows: tumor blood vessels are segmented based on the FITC-lectin signal. After blood vessel identification a “distance-map” is generated to allow the assignment to each pixel of the image a distinct distance value away from the closest blood vessel. Based on the “distance-map”, new objects are generated within the tumor, concentric to the blood vessels, and each is 10-μm wide. Finally, the average liposome MFI is calculated within each vessel-concentric object.

The mean liposome fluorescence intensity is determined by normalizing the liposome fluorescent signal within the tumor section by the area of the tumor section. :γ-H2AX stained tumor sections are analyzed in a similar fashion by segmenting the nuclei and subsequently classifying them into γ-H2AX positive or negative. The extent of cleaved PARP positive tumor cells is determined in cleaved PARP and cytokeratin double stained tumor sections. Tumor tissue is separated from background, nuclei are segmented based on the Hoechst® signal and cells are identified by growing the nuclei until reaching the edge of the cytokeratin signal. The cytokeratin signal is used to distinguish between tumor cells (cytokeratin positive) and non-tumor cells/stroma (cytokeratin negative). The tumor cells are then classified into PARP positive and PARP negative based on the intensity of the cleaved PARP staining.

### Example 1: Combination Therapy

Patients diagnosed with a HER2-positive cancer are treated with the combination of cyclophosphamide and MM-302 as follows:

As shown in Table E1, cyclophosphamide is administered at a dose of 600 mg/m² once every three weeks (Q3W) by intravenous injection over 60 minutes. MM-302 is then administered at a dose of 30 mg/m² Q3W by intravenous injection over 60 minutes. Cyclophosphamide is administered on day 1 of each cycle for the first four 3-week cycles. MM-302 is administered on day 2, day 3, day 4, day 5, or day 6 of each 3-week cycle.

TABLE E1

| Cyclophosphamide | MM-302 Dose |
|---|---|
| Dose (mg/m²) Q3W | (mg/m²) Q3W |
| 600 | 30 |
| Day 1 of cycle | Day 2-6 of cycle |

Patients diagnosed with a HER2-positive cancer are treated with the combination of cyclophosphamide, trastuzumab, and MM-302 as follows:

As shown in Table E2, cyclophosphamide is administered at a dose of 600 mg/m² Q3W by intravenous injection over 60 minutes. Trastuzumab is co-administered at a dose of 6 mg/kg Q3W (the first dose of trastuzumab is a loading dose of 8 mg/kg administered over 90 minutes followed by Q3W dosing at 6 mg/kg over 30-90 minutes via IV infusion). MM-302 is then administered at a dose of 30 mg/m² Q3W by intravenous injection over 60 minutes. Cyclophosphamide is administered on day 1 of each cycle for the first four cycles. Trastuzumab is administered on day 1 of each cycle and MM-302 is administered on day 6 of each cycle.

TABLE E2

| Cyclophosphamide | Trastuzumab Dose | MM-302 Dose |
|---|---|---|
| Dose (mg/m²) Q3W | (mg/kg) Q3W | (mg/m²) Q3W |
| 600 | 6 | 30 |
| Day 1 of cycle | Day 1 of cycle | Day 2-6 of cycle |

### Example 2: Patient Selection for Continuation of MM-302/Cyclophosphamide Combination Therapy

At the end of each cycle and just prior to dosing for subsequent cycles, neutrophil counts are checked. Administration of cyclophosphamide is not repeated in subsequent cycles if: a) the absolute neutrophil count is not greater than 1,500/mm³ and platelet count is not greater than 100,000/mm³; b) all non-hematologic toxicity (excluding alopecia) has resolved to ≦grade 1; c) hemorrhagic cystitis attributed to cyclophosphamide treatment is observed; and d) progression of disease is observed.

### Example 3: Reduced Doses for Combination Therapy comprising MM-302 and Cyclophosphamide

Patients receiving treatment comprising MM-302 and cyclophosphamide are monitored for toxicity. Modifications of MM-302 and cyclophosphamide dosing will be made using the dose levels shown in Table E3 below. Dose level 0 is the dose that patients receive during cycle 1. All toxicity will be graded according to the Common Toxicity Criteria (Version 4.0).

TABLE E3

| | MM-302 dose to | Cyclophosphamide dose to |
|---|---|---|
| Dose Level | be given | be given |
| 0 | 30 mg/m² | 600 mg/m² |
| −1 | 25 mg/m² | 450 mg/m² |
| −2 | 20 mg/m² | 300 mg/m² |

Patients who require a dose reduction of MM-302 and cyclophosphamide because of low nadir counts and mucositis (see below) will have the doses of MM-302 and cyclophosphamide reduced one dose level (not two dose levels).

### Nadir Counts:

The doses of MM-302 and cyclophosphamide will be permanently reduced by one dose level if the patient has Grade IV neutropenia: neutrophils/bands (<500/mm3) associated with fever requiring parenteral antibiotics, or Grade IV thrombocytopenia.

### Day 1 Counts:

ANC≧1500/mm³ and platelets≧50,000/mm³1: proceed with therapy.

ANC<1500/mm³: repeat CBC every 2 to 3 days until ANC≧1500/mm³.

Platelets <50,000/mm³: Therapy should be withheld until the platelets >50,000/mm³. If, however, the low platelet count is considered to be the result of bone marrow involvement, treatment should proceed. No changes in trastuzumab doses will be made for hematologic toxicity.

### Mucositis

Patients with grade ≧2 mucositis on day 1 of any treatment cycle should have their therapy delayed until the lesions have regressed to grade 1 or less. Patients may be delayed for up to 2 weeks. After 2 weeks, treatment will be stopped for any patient with persistent grade 2 or higher mucositis. MM-302 and cyclophosphamide should be permanently reduced one dose level if the patient develops grade 3 or 4 treatment induced mucositis. Patients who also concurrently require a dose reduction due to hematologic toxicity will have the MM-302 and cyclophosphamide doses reduced by only one level. Mucositis must have resolved to ≦grade 1 in order to proceed with day 1 treatment. No change in trastuzumab dosing will be made for mucositis. The dose should not be reduced if the mucositis is related to herpes simplex stomatitis.

### Hand-Foot Skin Reaction

Hand-Foot skin reactions will be graded according to the Common Toxicity Criteria (Version 4.0). No dose modification or delay in therapy is necessary for grade 1 toxicity. Patients with grade ≧2 palmar-plantar lesions on day 1 of any treatment cycle should have their therapy delayed until the lesions have regressed to grade 1 or less. Patients may be delayed for up to 2 weeks. For patients with grade 3 toxicity the dose of MM-302 should be reduced by 25%. After 2 weeks, any patient with persistent grade 2 or higher palmar plantar erythrodysesthesia will be removed from study treatment. No changes in the doses of cyclophosphamide or trastuzumab are required for palmar-plantar skin lesions.

### Liver Dysfunction

Doses of MM-302 should be modified according to the following schedule for any patient with an elevated direct (conjugated) bilirubin (DB)*:

DB≧5.0 mg/dl: The patient should not receive any further MM-302.

DB≧3.0-4.9 mg/dl: The patient should receive 25% of normal dose.

DB 1.2-3.0 mg/dl: The patient should receive 50% of normal dose.

*Dose adjustments must be made on the basis of direct bilirubin (DB).

At the discretion of the physician, G-CSF may be co-administered as dictated by the clinical situation.

### Example 4: Pretreatment with Cyclophosphamide Selectively Enhances Immunoliposome Delivery to Tumors

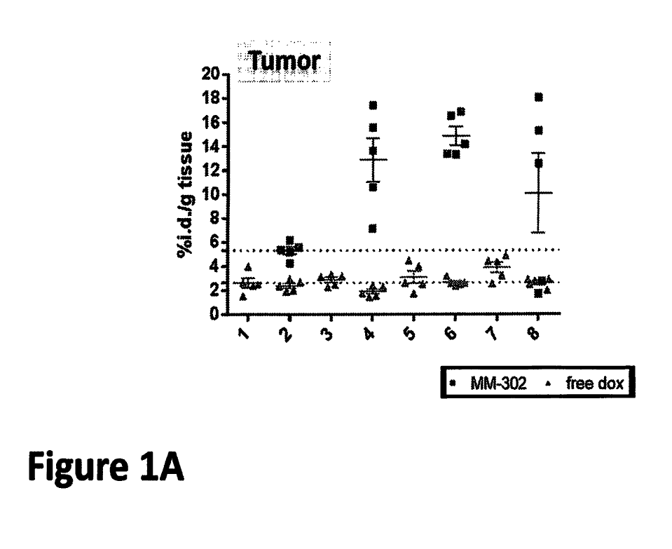

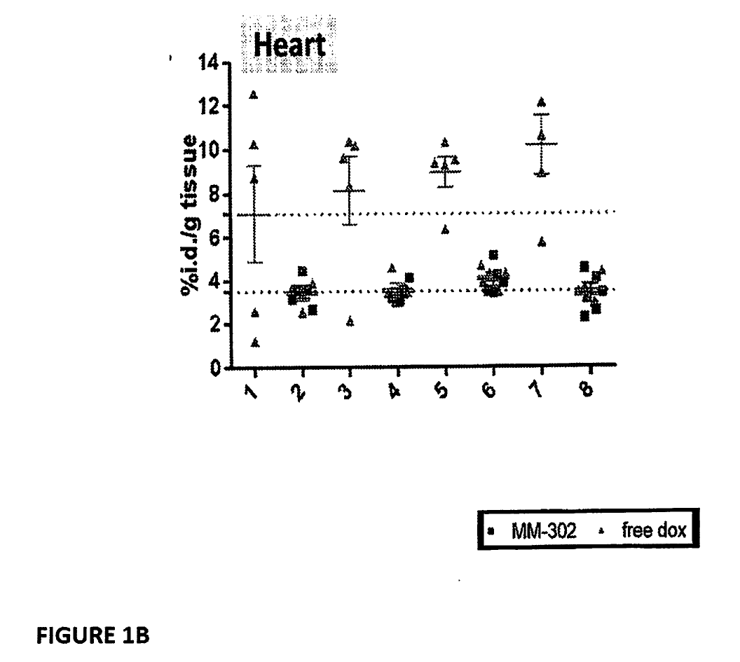

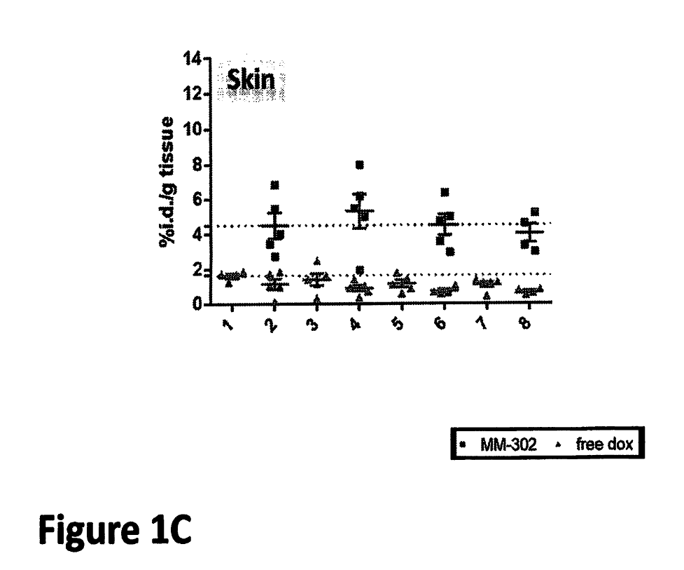

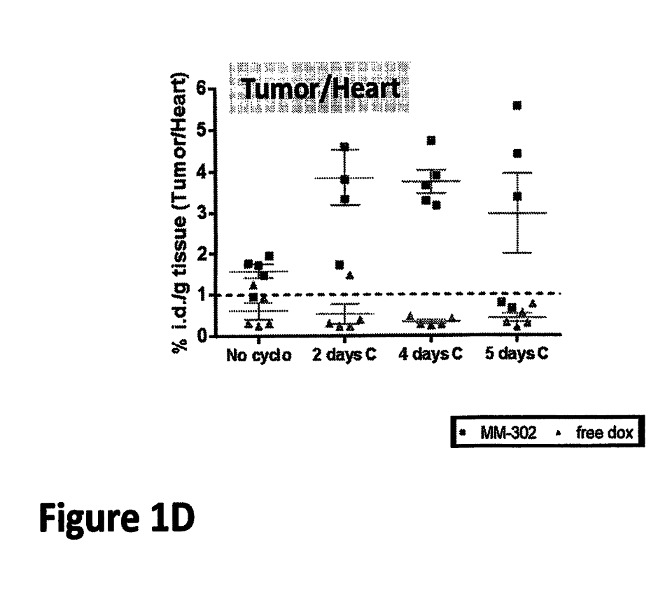

Mice were inoculated as described above with BT474-M3 breast cancer tumor cells, and were either untreated (no C), or pre-dosed with cyclophosphamide (170 mg/kg) 2, 4 or 5 days prior to MM-302 or free doxorubicin (both at 3 mg/kg) injection. Mice were sacrificed (24 hr post MM-302 or 30 min and 24 hr post free doxorubicin injection). Results are shown in FIGS. 1A-1D. The total doxorubicin content in tumors (FIG. 1A), hearts (FIG. 1B) or dorsal skin (FIG. 1C) was quantified by HPLC. The tumor/heart ratio for doxorubicin delivery is shown in (FIG. 1D). Pretreatment with cyclophosphamide significantly increased the amount of MM-302, but not free doxorubicin, that is deposited in the tumor (FIG. 1A) but not the heart (FIG. 1B). The effect of cyclophosphamide is tumor-specific and no effects were observed on non-target organs such as skin or heart.

### Example 5: Cyclophosphamide Pretreatment Enhances the Overall Tumor Exposure to MM-302

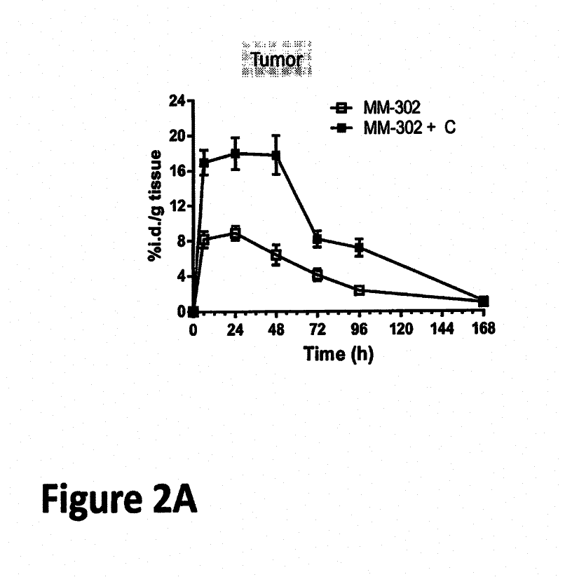

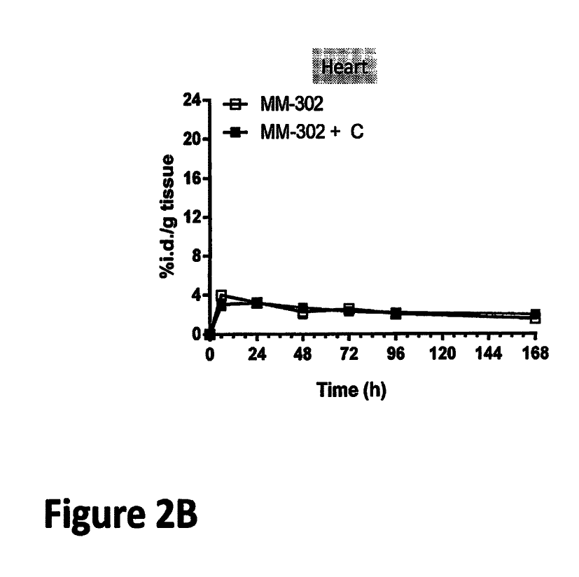

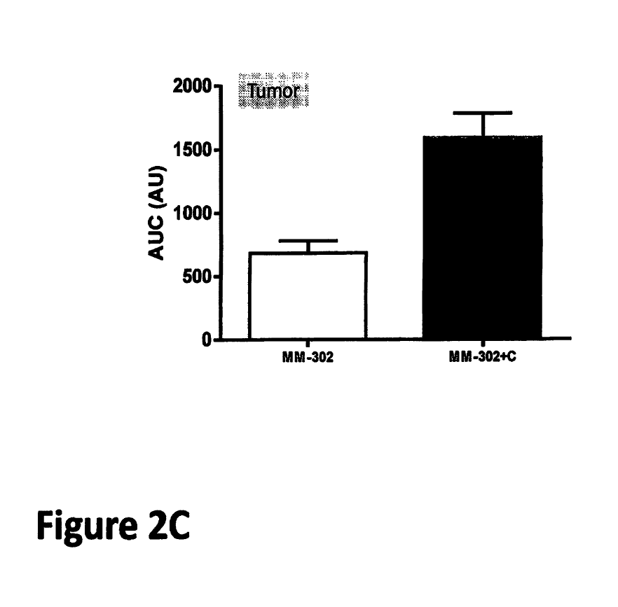

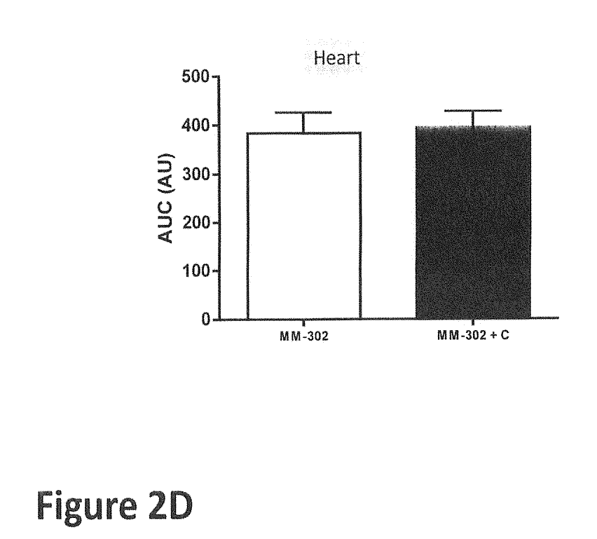

Mice were either untreated (PBS alone) or dosed with cyclophosphamide (170 mg/kg) 96 hours before injection of MM-302 (3 mg/kg). Mice were sacrificed at 6-168 hours post-MM-302 injection. As shown in FIGS. 2A-2D, tumors (FIG. 2A and FIG. 2C) and hearts (FIG. 2B and FIG. 2D) were excised and processed for doxorubicin quantification by HPLC. The areas under the curve (AUCs) with propagated error of the data in (FIG. 2A) and (FIG. 2B) were calculated for tumors (FIG. 2C) and hearts (FIG. 2D). Again, pretreatment with cyclophosphamide significantly increased exposure of the tumor to MM-302, but did not increase exposure of the cardiac tissue to the drug.

### Example 6: Cyclophosphamide Pretreatment Induces Tumor Cell Apoptosis, Reduces Tumor Cell Density, and Increases the Interstitial Space Following Immunoliposome Injection

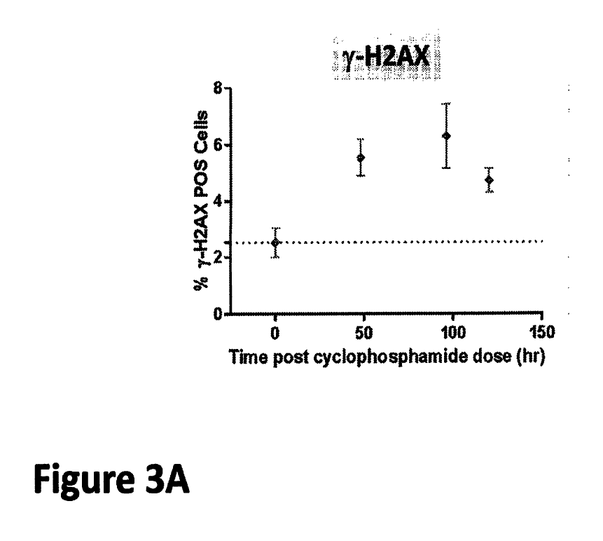

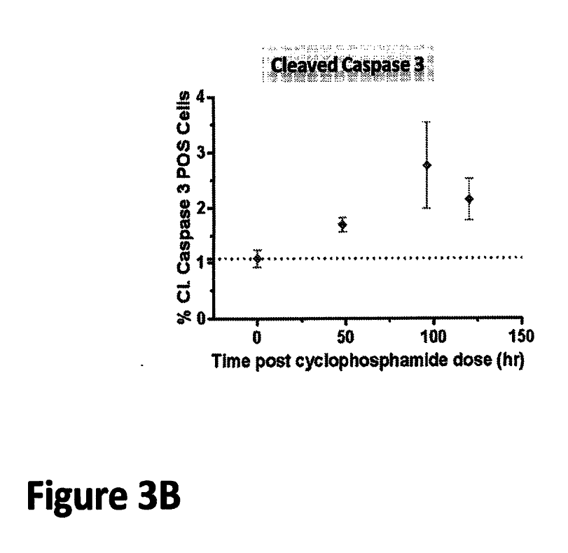

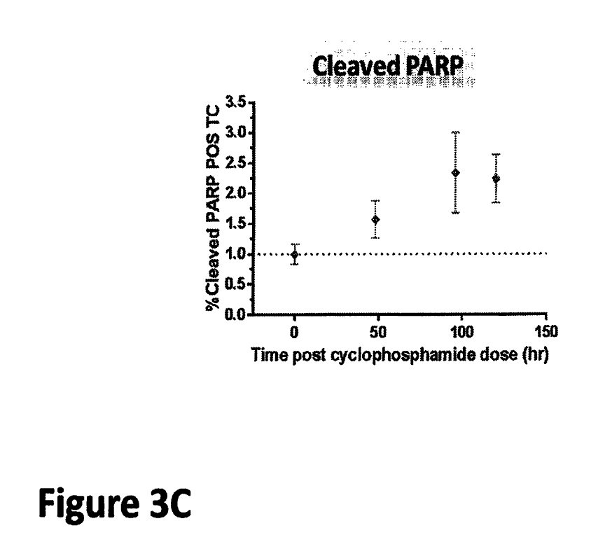

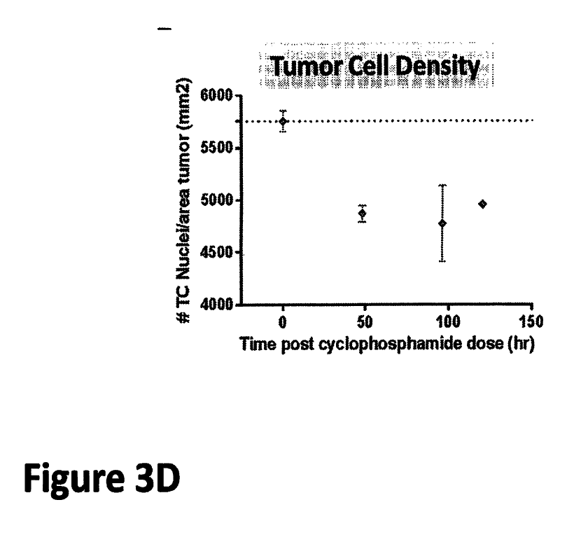

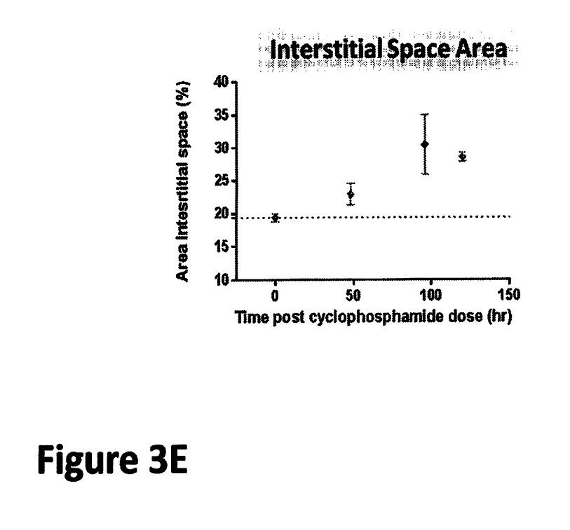

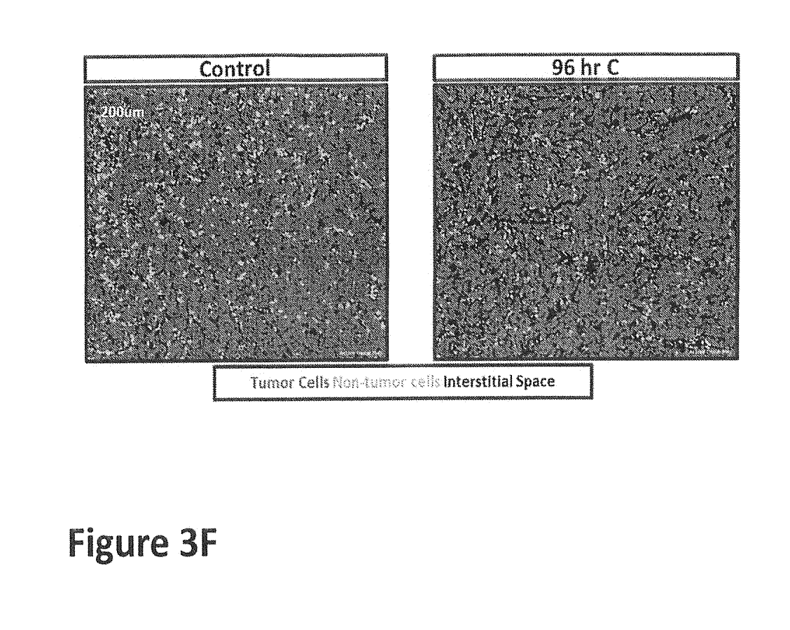

Tumor tissue sections from cyclophosphamide-treated mice (tissue harvested at 48 hr-120 hr post treatment) were stained with an anti-γ-H2AX and anti-cleaved caspase 3 antibody mix (FIGS. 3A and 3B) or an anti-cleaved PARP and anti-pan human cytokeratin antibody mix (FIG. 3C). Slides were scanned and images were analyzed with Definiens® Developer XD to quantify the % of γ-H2AX positive cells (FIG. 3A), cleaved caspase 3 positive cells (FIG. 3B) or cleaved-PARP positive tumor cells (FIG. 3C). The number of tumor cell nuclei per tumor area (mm²) (FIG. 3D) and the area of the interstitial space (%) (FIG. 3E) were quantified from the images in (FIG. 3C). Representative fields of view after image analysis are shown in (FIG. 3F). These data indicate that induction of tumor cell apoptosis and consequent reduction of tumor cell density and increase in interstitial space are among the mechanisms responsible for the increase in MM-302 delivery.

### Example 7: Cyclophosphamide Enhances Nuclear Delivery of Doxorubicin Upon Immunoliposome Injection, with a Resulting Increase in DNA-Damage and Apoptosis

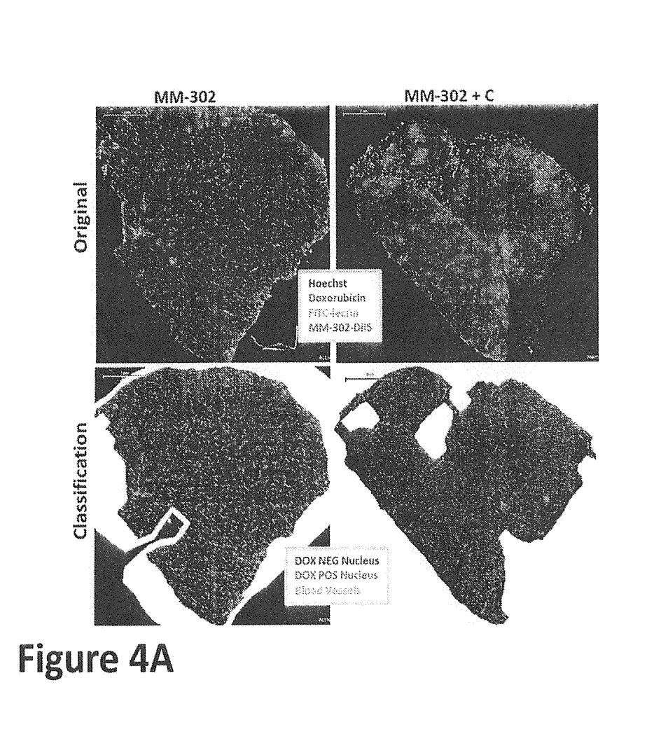

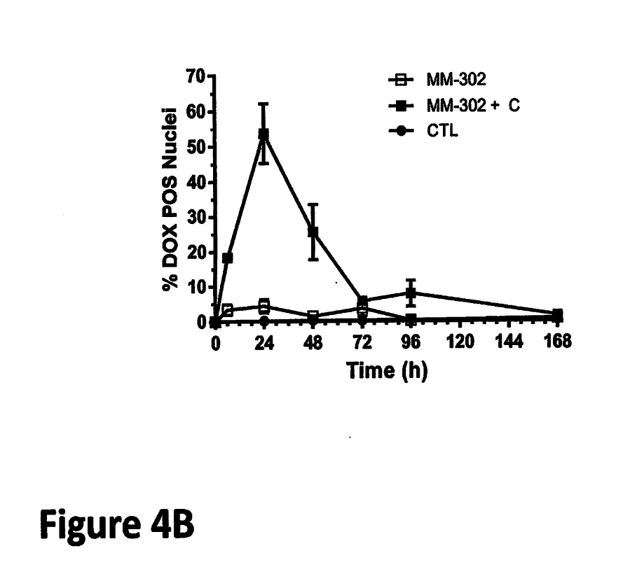

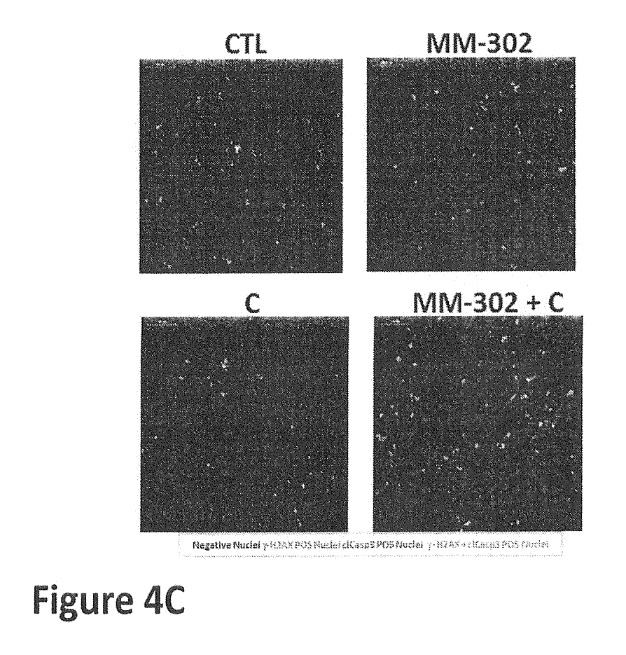

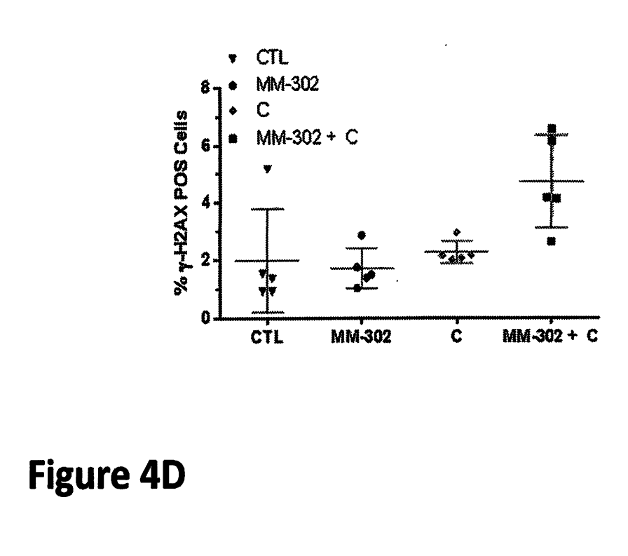

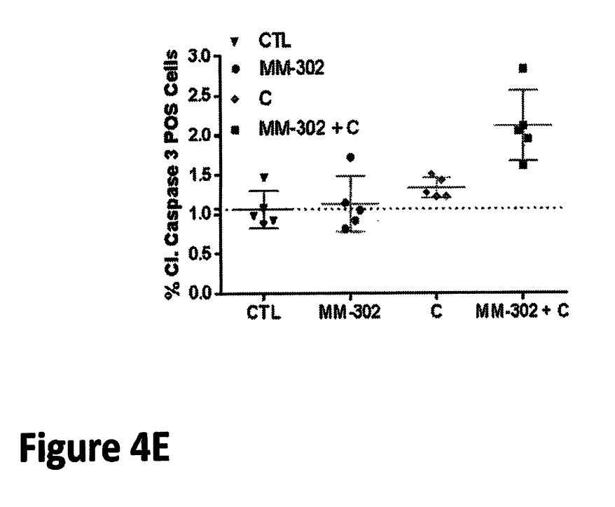

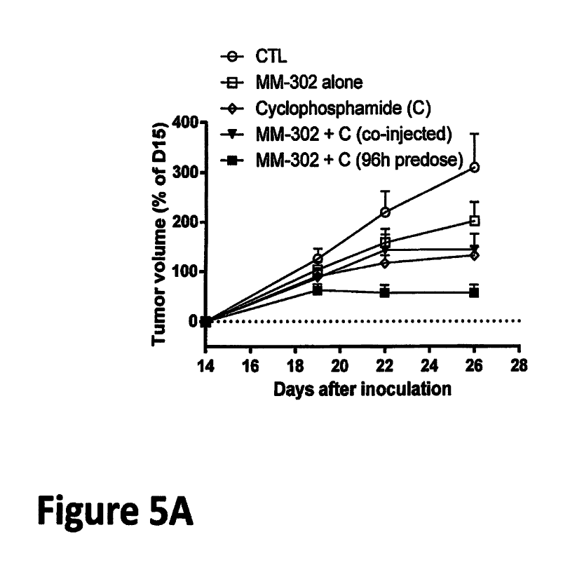

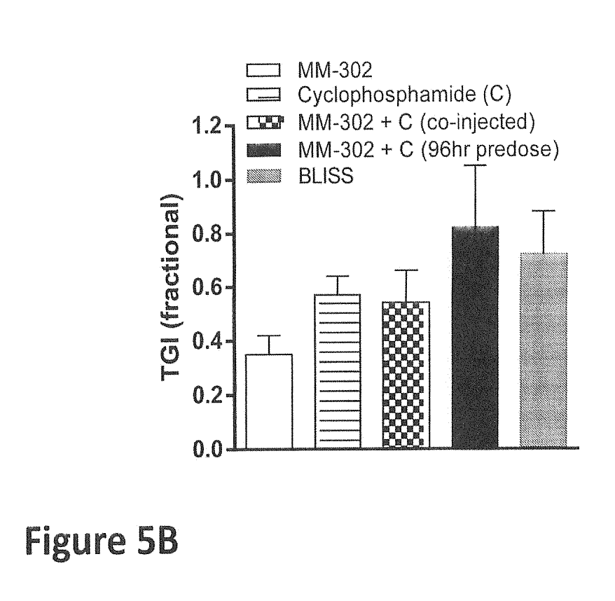

BT474-M3 tumor-bearing mice were untreated or dosed with cyclophosphamide (C) 96 hr before injection of MM-302. Mice were sacrificed 24 hr post-MM-302 injection and tumors were collected. DiI5-labelled MM-302 (MM-302-DiI5), doxorubicin, FITC-lectin labeled blood vessels and nuclei were imaged on frozen sections. Images were analyzed with Definiens Developer XD and results are shown in FIGS. 5A and 5B. Representative tumors are shown (FIG. 4A) as original images (top panels) and post-classification (bottom panels). The quantification of doxorubicin positive nuclei is shown in (FIG. 4B). Tumor tissue sections were stained with an anti-γ-H2AX and anti-cleaved caspase 3 antibody mix. Slides were scanned, and the images were analyzed with Definiens® Developer XD to quantify the % of γ-H2AX and cleaved caspase 3 positive cells. Representative fields of view post-analysis are shown in (FIG. 4C) and the results of the quantification are shown in (FIG. 4D) and (FIG. 4E), for γ-H2AX and cleaved caspase 3, respectively. Thus, the increase in MM-302 delivery to tumors is accompanied by an increase in doxorubicin accumulation to the nuclei of tumor cells and subsequent activation of downstream markers of DNA damage/repair and apoptosis.

### Example 8: Cyclophosphamide Enhances Nuclear Delivery of Doxorubicin Upon Immunoliposome Injection, with a Resulting Increase in DNA-Damage and Apoptosis

As shown in FIGS. 5A-5B, BT474-M3 tumor-bearing mice were either untreated (CTL, open circles) or treated with MM-302 alone (open squares), cyclophosphamide (C, open diamonds), or a combination of the two agents, co-injected (solid triangles), or with C given 96 hr prior to MM-302 (solid squares). Tumor volume was measured over time starting at 14 days after inoculation. The % change in tumor growth relative to the first day of treatment (day 15) is shown in FIG. 5A. FIG. 5B shows Bliss Independence Analysis at day 26. As shown in this and the preceding examples, pre-dosing of BT474-M3 tumors with cyclophosphamide followed by administration of MM-302 results in improved anti-tumor activity compared to either single agent alone or co-administration of the two agents.

### Example 9. Cyclophosphamide Enhances Tumor Deposition of Immunoliposomes Regardless of Route of Administration

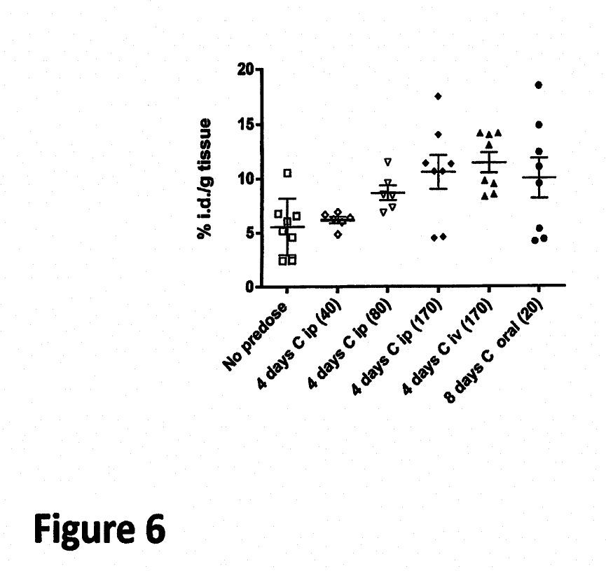

Mice were inoculated with BT474-M3 breast cancer tumor cells (15×10⁶ cells were injected into the left and right mammary fat pad). When the tumor volume reached between 200 and 300 mm³, mice were injected with MM-302 alone (empty black squares), or were pre-treated with cyclophosphamide followed by MM-302 according to the following regimens: 40 mg/kg cyclophosphamide i.p. (empty diamonds), 80 mg/kg i.p. (empty lower triangles), 170 mg/kg i.p. (filled diamonds), 170 mg/kg i.v. (filled upper triangles) 4 days prior to MM-302. In addition, eight consecutive daily doses of cyclophosphamide at 20 mg/kg were given by oral gavage (filled circles) starting 1 week prior MM-302 injection. Mice were sacrificed 24 h post MM-302 injection and tumors were collected for quantification of doxorubicin by HPLC. (n=3-4 mice group with two tumors/mouse). Data are represented as the % injected MM-302 dose per gram of tumor tissue as measured by the amount of doxorubicin in the tumor tissue. As shown in FIG. 6, cyclophosphamide enhances tumor deposition of MM-302/doxorubicin regardless of the route of administration.

### Example 10: Pretreatment with Cyclophosphamide Enhances Tumor Penetration of Targeted Immunoliposomes

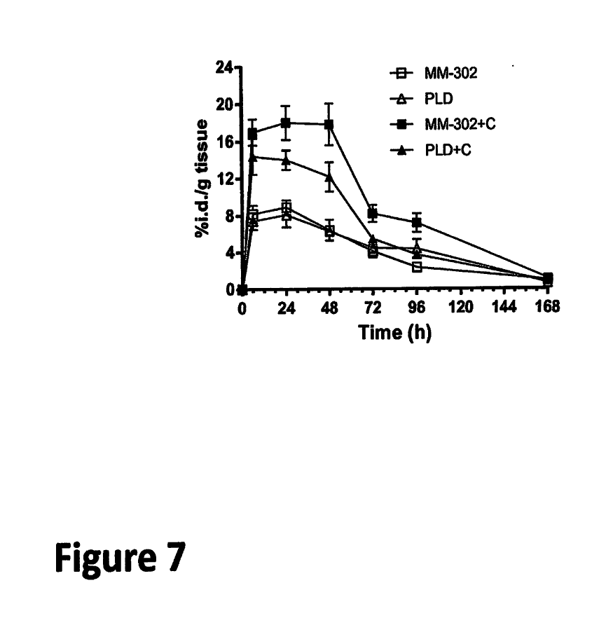

Mice were inoculated with BT474-M3 breast cancer tumor cells (15×10⁶, into the mammary fat pad). When the tumor volume reached between 200 and 300 mm³, mice were injected with either MM-302 (empty squares) or pegylated liposomal doxorubicin (PLD—untargeted MM-302) (empty upper triangles) (both at 3 mg/kg dox equivalents). Alternatively, mice received a single dose of cyclophosphamide (170 mg/kg, i.p.) given 4 days prior to MM-302 (filled squares) or PLD (filled upper triangles). Individual groups of mice (n=5 mice group) were sacrificed at 6 h, 24 h, 48 h, 72 h, 96 h or 168 h post MM-302 of PLD injection, and tumors were collected for quantification of doxorubicin by HPLC. As shown in FIG. 7, tumors from mice that were pretreated with cyclophosphamide followed by administration of MM-302 had a significantly higher percentage of the injected dose of doxorubicin in the tumor tissue than mice treated with MM-302 alone. Surprisingly, in mice pretreated with cyclophosphamide, the tumors from mice that were administered targeted liposomes showed a significantly higher amount of doxorubicin than tumors from mice that were administered untargeted liposomes (PLD). These data suggest that pretreatment with cyclophosphamide, combined with the greater retention capacity of the targeted liposomes, results in a significant increase in exposure of the tumor to the anthracycline.

### Example 11: Clinical Data Demonstrating that Pre-Dosing with Cyclophosphamide Enhances Immunoliposome Deposition and Provides Increased Clinical Benefits

MM-302 was labeled by a commercial radiopharmacy with ⁶⁴Cu (obtained from Washington University in St. Louis) using a gradient-loadable chelator, 4-DEAP-ATSC. For positron emission tomography (PET) imaging at cycle 1, patients (n=12 to date) with HER2-positive metastatic breast cancer received 30 mg/m² of MM-302 followed by a trace dose of ⁶⁴Cu-MM-302 (3-5 mg/m², 400 MBq). From cycle 2 and on, patients continue to receive MM-302 (30 mg/m², q3w) until disease progression; disease response is monitored every 8 weeks following RECIST 1.1 guidelines. In addition to MM-302, a subset of patients received 6 mg/kg of trastuzumab (q3w) 5 days prior to MM-302 treatment; another subset of patients received 450 mg/m² of cyclophosphamide (q3w, first 4 cycles) and trastuzumab (6 mg/kg, q3w) 5 days prior to MM-302.

PET/CT (computed tomography) images were acquired post-⁶⁴Cu-MM-302 injection (Day 1, Scan 1, <3 hours), and on Day 2 (Scan 2) or Day 3 (Scan 3), or on all 3 days. Diagnostic ¹⁸F-FDG-PET/CT or CT images, where available, were used to identify additional lesions that have low ⁶⁴Cu-MM-302 uptake. Average tumor deposition was quantified by region of interest (ROI) analysis using MIM Software (version 6.2), expressed as percentage of injected dose per kilogram of tissue (% i.d./kg) derived from median standardized uptake values (SUV_{median}; which was found to be similar to SUVₘₑₐₙ).

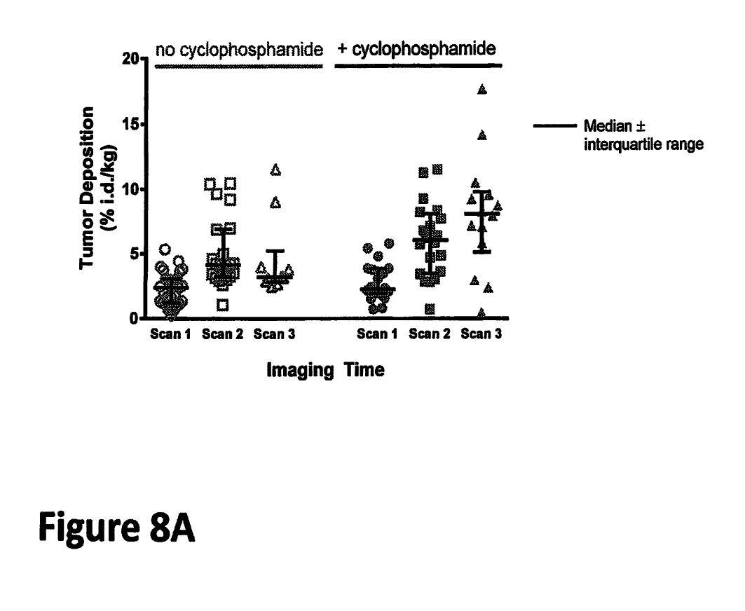

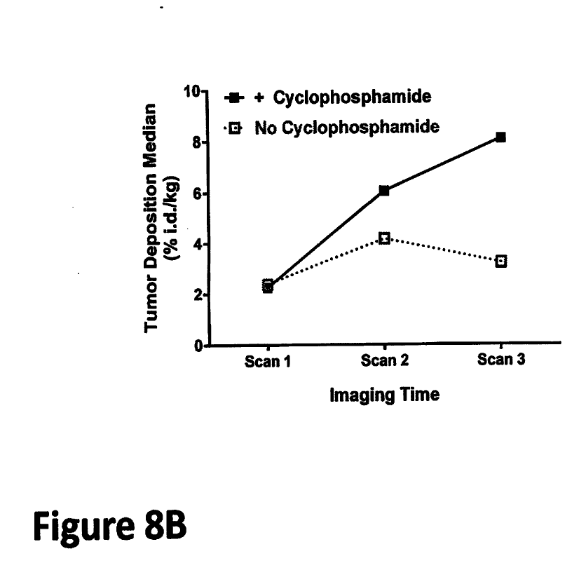

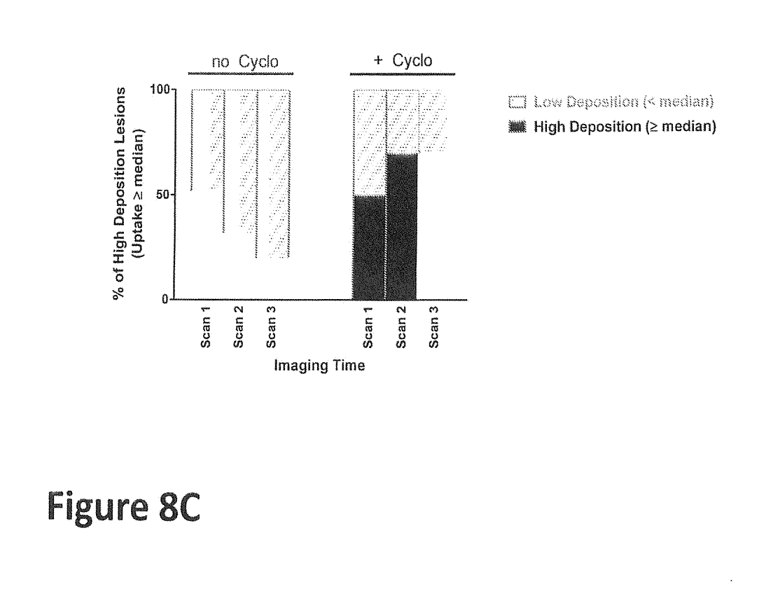

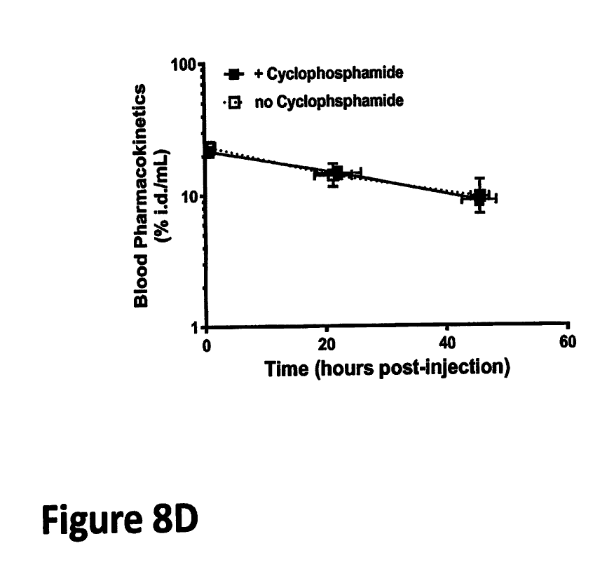

FIG. 8A illustrates the tumor deposition in patients pre-treated with and without cyclophosphamide. In both groups, tumor deposition was found to be variable within each patient, and across different patients. FIG. 8B shows that the median tumor deposition on Days 2 and 3 in patients treated with cyclophosphamide was higher than in patients who did not receive cyclophosphamide, while baseline tumor uptake (predominantly from ⁶⁴Cu-MM-302 in tumor vasculature) is similar in the 2 groups of patients.

The overall tumor deposition median for each scan day was used to establish a pseudo-threshold to identify tumors (lesions) with low (<median) and high (≧median)⁶⁴Cu-MM-302 deposition. FIG. 8C shows that more high deposition tumors were identified in patients who were pre-treated with cyclophosphamide on Scans 2 and 3 (primarily representing tissue-deposited drugs). Note that this deposition enhancement effect of cyclophosphamide is specific to the tumors, with no significant difference observed in the drug exposure (blood pharmacokinetics, FIG. 8D) between patients treated with or without cyclophosphamide.

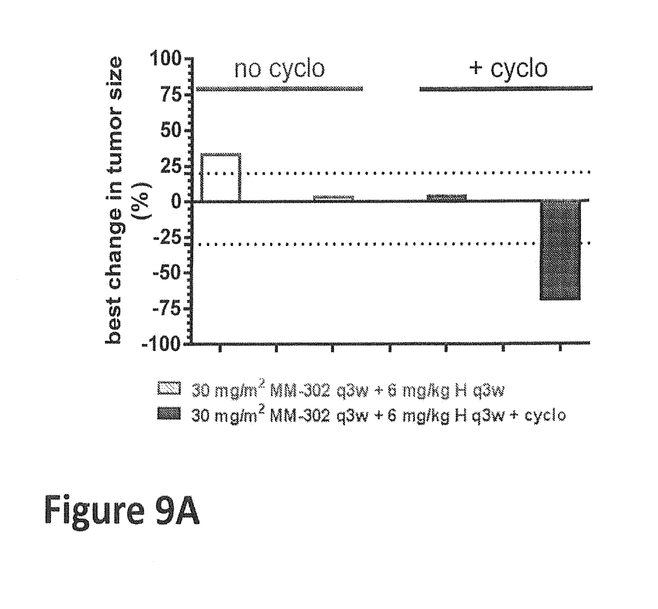

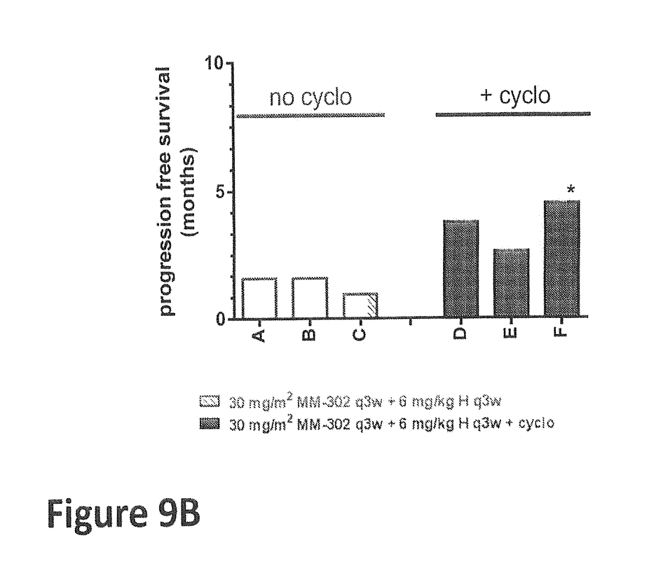

Early assessment of response was measured by both change in tumor size (FIG. 9A) and progression free survival (FIG. 9B). Results that were obtained suggest that the patient group treated with cyclophosphamide (MM-302+ trastuzumab+ cyclophosphamide) achieved superior clinical outcomes compared to those without cyclophosphamide (MM-302+ trastuzumab).

### Example 12: Pretreatment with Carboplatin Increases the Deposition of EphA2 Targeted Immunoliposomes in the Tumor

Mouse xenograft studies were performed using the following cell types: Calu3 (Lung, ATCC® HTB-55™), H2170 (lung, ATCC® CRL5928™), H522 (lung, ATCC® CRL-5810™), Skov3 (Ovarian, ATCC® HTB-77™), and SUM149 (breast, Asterand). Tumor-bearing animals were treated with carboplatin or saline for 96 hours prior to administration of fluorescently labeled EphA2 targeted (i.e., comprising externally oriented anti-EphA2 scFv antibodies as described in US patent publication No. 20130209481) immunoliposomes. Animals were sacrificed 72 hours after the immunoliposomes were administered, and liver, spleen and tumor were frozen in optimal cutting temperature (OCT) compound for assessment of liposome microdistribution in the tissue using fluorescent microscopy. A set of tissue microarrays was generated from the experiment which included, on each slide, tumor, liver and spleen samples of saline and carboplatin-pre-treated animals to minimize microscopy related variability. Slides were fixed in 4% paraformaldehyde, coverslipped with Prolong® gold and scanned using an Aperio® FL scanner. Images were analyzed using an in-house algorithm built with MATLAB software from The MathWorks®.

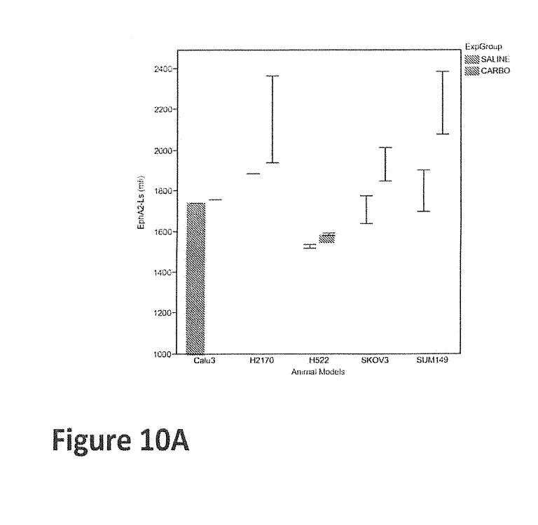

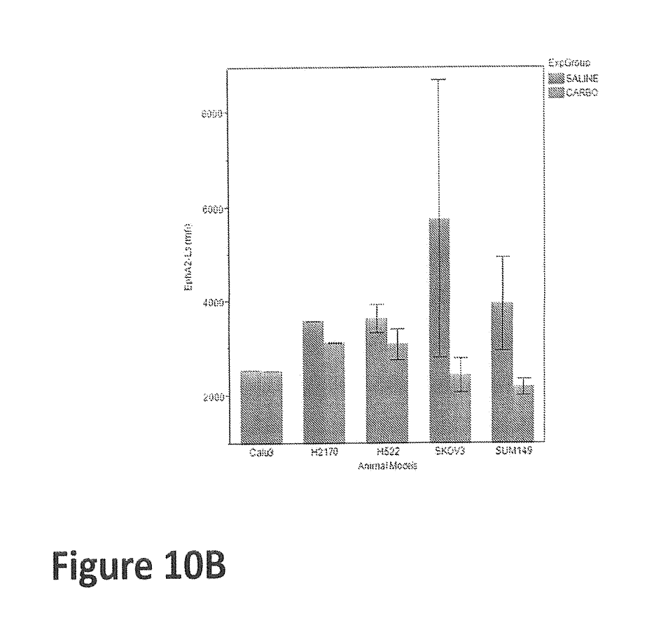

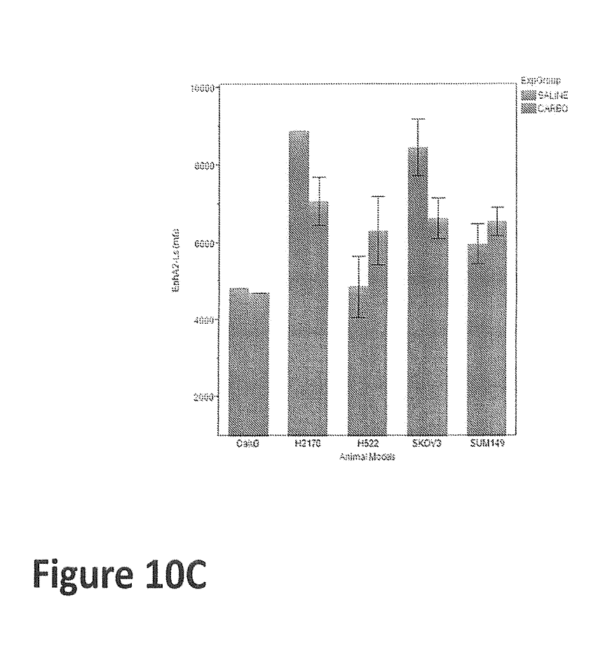

Mean fluorescence intensity (mfi) was calculated for each tissue area. Mean and SEM were plotted. Data for each cell line are presented pairwise with saline-treated animals represented by the left hand bar, and carboplatin-treated animals by the right hand bar. As shown in FIG. 10A, carboplatin-treated animals had a significantly immunoliposome concentration in tumor compared to saline treated animals. Conversely, liposome deposition in the liver (FIG. 10B) and spleen (FIG. 10C) was not increased in response to carboplatin pretreatment; such organ samples from most animals showed a decrease in liposome deposition compared to saline treated animals.

### EQUIVALENTS

Those skilled in the art will recognize, or be able to ascertain and implement using no more than routine experimentation, many equivalents of the specific embodiments described herein. Such equivalents are intended to be encompassed by the following claims. Any combination, or combinations, of the embodiments disclosed in the dependent claims are contemplated to be within the scope of the disclosure.

### INCORPORATION BY REFERENCE

The disclosure of each and every U.S. and foreign patent and pending patent application and publication referred to herein is specifically incorporated by reference herein in its entirety.

1.-13. (canceled)

14. A method of treating a cancer in a human patient, the method comprising at least one treatment cycle, each cycle comprising administration of an organoplatinum agent to the patient followed by administration of a therapeutically effective amount of an immunoliposome comprising an encapsulated chemotherapeutic agent and a plurality of externally oriented antibody molecules, wherein the administration of the immunoliposome is parenteral and is initiated from two to ten days after initiation of the administration of the organoplatinum agent.

15. The method of claim 14, wherein the organoplatinum agent is carboplatin.

16. The method of claim 14, wherein, in each cycle, the administration of the immunoliposome is initiated about six days, about seven days, or about eight days after the administration of the organoplatinum agent is initiated.

17. The method of claim 16, wherein, in each cycle, the administration of the immunoliposome is initiated about seven days after the administration of the organoplatinum agent is initiated.

18. The method of claim 14, wherein the administration of the organoplatinum agent is parenteral administration.

19. The method of claim 18 wherein, in each cycle, the parenteral administration of the organoplatinum agent is a single intravenous administration.

20. The method of claim 14, wherein the antibody molecules bind immunospecifically to a human cell surface receptor that is an ephrin receptor.

21. The method of claim 20, wherein the ephrin receptor is EphA2.

22. The method of claim 14, wherein the plurality of antibody molecules consists of 10-200 scFv molecules.

23. The method of claim 14, wherein the chemotherapeutic agent is selected from the group consisting of 2-chloro adenosine, 5-azacytosine, 5-azacytosine-arabinoside, 5′-deoxyfluorouridine, 5-FU, 5-imidodaunomycin, 6-mercaptopurine, allopurinol, aminoglutethimide, aminopterin, anastrozole, azathioprine, bicalutamide, bleomycin, bryostatin, busulfan, capecitabine, carboplatin, carcinomycin, carmustine, chlorambucil, cisplatin, cladribine, cyclophosphamide, cytosine arabinoside, dacarbazine, dactinomycin, daunorubicin, daunoryline mitoxantrone, deoxycytidine, didanosine, diethylstilbestrol, docetaxel, cabazitaxel, doxorubicin, droloxifene, edatrexate, epirubicin, estradiol, etoposide, finasteride, fludarabine, fluorodeoxyuridine, flutamide, ftorafur, gemcitabine, hydroxyurea, idarubicin, ifosfamide, irinotecan, leuprolide, lomustine, lurtotecan, mechlorethamine, medroxyprogesterone acetate, megesterol acetate, melphalan, methotrexate, mitomycin, mitotane, N-acetyladriamycin, N-acetyldaunomycine, ormaplatin, oxaliplatin, paclitaxel, pegaspargase, pentostatin, pentostatin, perfosfamide, pirarubicin, platinum-DACH, plicamycin, pyrimethamine, pyritrexim, rubidazone, rubidomycin, silatecan, streptozocin, streptozocin, tamoxifen, teniposide, testolactone, tetraplatin, thioguanine, thiotepa, thymitaq, tolmudex, topotecan, toremefine, trimethoprim, trimetrexate, trioxifene, trophosphamide, vinblastine, vincristine, vindesine, vinflunine, vinorelbine, vinpocetine, and zalcitabine.

24. The method of claim 14, wherein the treatment cycle is a three-week treatment cycle.

25. The method of claim 14, wherein the cancer is a solid tumor.

26. The method of claim 25, wherein the solid tumor is an ovarian tumor.
